(19) United States
(12) Patent Application Publication
(10) Pub. No.: US 2026/0328777 A1
(43) Pub. Date: Oct. 22, 2026

(54) MITIGATION OF CHECKPOINT INHIBITOR-INDUCED AUTOIMMUNE HEMOLYTIC ANEMIA THROUGH MODULATION OF PURINERGIC SIGNALING

(71) Applicant: THE TRUSTEES OF COLUMBIA UNIVERSITY IN THE CITY OF NEW YORK, New York, NY (US)

(72) Inventors: Krystalyn E. HUDSON, Cliffside Park, NJ (US); Flavia Dei ZOTTI, New York, NY (US)

(21) Appl. No.: 19/574,966

(22) Filed: Mar. 23, 2026

Related U.S. Application Data
(63) Continuation-in-part of application No. PCT/US2024/049041, filed on Sep. 27, 2024.
(60) Provisional application No. 63/585,928, filed on Sep. 27, 2023.
(60) Provisional application No. 63/674,741, filed on Jul. 23, 2024.

(51) Int. Cl. G01N 33/68; A61K 38/46

(52) U.S. Cl. CPC G01N 33/6869; A61K 38/46; G01N 33/6893; G01N 2800/24

(57) ABSTRACT

A new T cell population that predicts onset of autoimmune hemolytic anemia was identified in preclinical mouse models of autoimmune hemolytic anemia (AIHA) and in patients with AIHA. The cell phenotype is CD4⁺CD39⁺CD73⁻CD25⁻FoxP3⁻, and these cells secrete IL-17 and/or IFNy upon stimulation, which perpetuates autoimmune pathology and is a marker for determining the onset of an immune-related adverse event. The preclinical mouse models of AIHA also revealed that prophylactic/concurrent apyrase treatment mitigates disease onset by countering modulation of the purinergic signaling pathway caused by failure of tolerance to red blood cells.

### CROSS-REFERENCE TO RELATED APPLICATIONS

This application is a continuation-in-part application of International Application No. PCT/US2024/049041, filed on Sep. 27, 2024, which claims the benefit of U.S. provisional patent application 63/585,928, filed Sep. 27, 2023, to The Trustees of Columbia University, titled “NEW BIOMARKER AND TREATMENT FOR AUTOIMMUNE HEMOLYTIC ANEMIA (AIHA),” and U.S. provisional patent application 63/674,741, filed Jul. 23, 2024, to The Trustees of Columbia University, titled “MITIGATION OF CHECKPOINT INHIBITOR-INDUCED AUTOIMMUNE HEMOLYTIC ANEMIA THROUGH MODULATION OF PURINERGIC SIGNALING,” the entirety of the disclosures of which are hereby incorporated by this reference.

### STATEMENT REGARDING FEDERALLY SPONSORED RESEARCH OR DEVELOPMENT

This invention was made with government support under HL133325 awarded by the National Institutes of Health. The government has certain rights in the invention.

### TECHNICAL FIELD

The subject matter disclosed herein is generally directed to detecting and treating immune checkpoint inhibitor (ICPi) induced immune-related adverse events (irAEs).

### BACKGROUND

Immune checkpoint inhibitors (ICPi) have redefined the landscape of cancer immunotherapy by blocking pathways that dampen immune responses to tumor antigens(1-3). Because these pathways mediate immune tolerance, ICPi also induce autoimmune pathologies in some patients(4). Immune-related adverse events (irAEs) can affect multiple organs and systems. The most frequently-reported irAEs in the hematologic compartment is the induction of autoimmune hemolytic anemia (AIHA) that can result in severe hemolysis and high mortality rates(5-7). Although the cause of irAEs seems obvious (i.e., checkpoint inhibition), the underlying immunological mechanisms of AIHA secondary to ICPi therapy (ICPi-AIHA) has been largely unexplored.

In studies with red blood cell (RBC) transgenic mice, we previously reported that T cell tolerance is required to prevent AIHA(8). In addition, unlike most autoantigens, tolerization of RBC autoreactive T cells does not occur in the thymus; instead, autoreactive CD4 recent thymic emigrants (RTEs) encounter RBCs in the periphery and upregulate expression of inhibitory receptors (e.g., PD1) along with transcription factors associated with anergy, exhaustion, and regulatory T cells (Tregs)(9). Although these events together produce stringent tolerance to RBC autoantigens, they are frequently abrogated in females and upon aging, as seen in human patients presenting with AIHA(10, 11).

Citation or identification of any document in this application is not an admission that such a document is available as prior art to the present invention.

### SUMMARY

The current studies developed a new model of AIHA secondary to ICPi therapy. We demonstrate that ICPi targeting the specific pathways associated with RTE tolerance to RBC autoantigens caused decreases in two Treg populations (FoxP3⁺ and Tr1) and expansion of pro-inflammatory Th17 T cells. Simultaneously, a novel CD4⁺ T cell subset which expresses CD39 (i.e., CD39 single positive [CD39SP]) emerged; the presence of CD39SP predicted subsequent AIHA development in ICPi-treated mice. This model of ICPi-induced tolerance failure to RBC autoantigens, accompanied by the appearance of CD39SP, also occurred in both lupus-prone NZB mice and healthy wild-type B6 mice. Finally, as CD39 functions as an ATP hydrolase, injection of apyrase to supplement CD39's enzymatic activity (i.e., ectonuclease) mitigated ICPi-AIHA.

Together, the findings establish a causal role for specific immunoregulatory molecules in RBC autoantigen tolerance, generate a novel model for a major sequela of immune checkpoint inhibitor therapy, identify a new subset of T cells that predicts AIHA development, and introduce a novel therapeutic intervention. Although each of these findings were generated in murine models, we report the novel finding that humans with AIHA have increased CD39SP compared to healthy controls, supporting the potential translatability of these findings.

In one aspect, the present invention provides for a method of determining the onset of an immune-related adverse event (irAE) in a subject, the method comprising: providing a blood or serum sample from the subject; determining the presence of cells in the blood or serum sample that express CD4 and CD39; and determining the presence of blood cells that lack expression of CD73, CD25, and FoxP3 in the blood cells that express CD4 and CD39, wherein the presence of such blood cells indicates increased likelihood for the onset of an irAE. In certain embodiments, the method further comprises determining the level of IL-17 and/or IFNy secretion in the blood cells that express CD4 and CD39 and lack expression of CD73, CD25, and FoxP3 from the blood or serum sample, wherein secretion of IL-17 and/or IFNy confirms increased likelihood for the onset of an irAE. In certain embodiments, the irAE is autoimmune hemolytic anemia.

In another aspect, the present invention provides for a method of preventing the onset of an immune-related adverse event (irAE) in a subject, the method comprising administering to the subject a therapeutic that degrades ATP. In certain embodiments, the therapeutic that degrades ATP is apyrase. In certain embodiments, the apyrase is recombinant apyrase. In certain embodiments, the method further comprises identifying the subject is at an increased likelihood of developing an irAE prior to administering apyrase. In certain embodiments, the step of identifying the subject is at an increased likelihood of developing an irAE comprises: providing a blood or serum sample from the subject; determining the presence of cells in the blood or serum sample that express CD4 and CD39; and determining the presence of blood cells that lack expression of CD73, CD25, and FoxP3 in the blood cells that express CD4 and CD39, wherein the presence of such blood cells indicates increased likelihood for the onset of an irAE. In certain embodiments, the method further comprises determining the level of IL-17 and/or IFNy secretion in the blood cells that express CD4 and CD39 and lack expression of CD73, CD25, and FoxP3 from the blood or serum sample, wherein secretion of IL-17 and/or IFNy confirms the subject is at an increased likelihood of developing an irAE. In certain embodiments, the irAE is autoimmune hemolytic anemia. In certain embodiments, the subject is administered an immune checkpoint inhibitor (ICPi) before, after, or concurrently with the therapeutic that degrades ATP. In certain embodiments, the ICPi is selected from the group consisting of anti-TIM3, anti-CTLA4, anti-PD-L1, anti-PD1, anti-TIGIT, and anti-LAG3.

In another aspect, the present invention provides for a method of identifying a potential therapeutic for preventing and/or treating autoimmune hemolytic anemia comprising: inducing autoimmune hemolytic anemia by administering an immunotherapy composition to a mammalian subject comprising: a blocking antibody against PD1; a blocking antibody against Lag-3; a blocking antibody against CTLA-4; and a blocking antibody against IL-10 receptor; detecting percentage of a subset of T cells in the mammalian subject, wherein the subset of T cells is positive for CD39 and negative for CD73, FoxP3, and CD25; and administering the potential therapeutic to the mammalian subject. In certain embodiments, the subset of T cells is CD4 T cells. In certain embodiments, the percentage of the subset of T cells in the mammalian subject is detected a first time before administration of the potential therapeutic but after administration of the immunotherapy composition and a second time after administration of the potential therapeutic. In certain embodiments, the method further comprises detecting the presence of autoantibodies in the mammalian subject. In certain embodiments, the mammalian subject is murine.

In another aspect, the present invention provides for a method of treating an immune-related adverse event (irAE) in a subject, the method comprising reducing the percentage of a subset of T cells positive for CD39 and negative for CD73, FoxP3, and CD25 in the subject. In certain embodiments, the subject is administered a therapeutic that degrades ATP thereby reducing the percentage of the subset of T cells positive for CD39 and negative for CD73, FoxP3, and CD25 in the subject. In certain embodiments, the therapeutic that degrades ATP is apyrase. In certain embodiments, the apyrase is recombinant apyrase. In certain embodiments, the method further comprises administering a steroid and/or rituximab to the subject. In certain embodiments, the irAE is autoimmune hemolytic anemia. In certain embodiments, the irAE is induced by inhibition of an immune checkpoint. In certain embodiments, the immune checkpoint is selected from the group consisting of: PD1 activation, Lag-3 activation, CTLA-4 activation, and IL-10 receptor activation.

The foregoing and other aspects, features, and advantages will be apparent from the DESCRIPTION and DRAWINGS, and from the CLAIMS.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present invention will hereinafter be described in conjunction with the appended and/or included DRAWINGS, where like designations denote like elements, and:

FIG. 1—RBC autoreactive RTE tolerization involves checkpoint molecules and Tregs. CD4⁺ T cells from 10-12-week-old HODxOTII F1 mice were sorted for RNAseq. (A) Heatmap representing differences of transcript expression in CD4⁺ T cells from autoreactive HOD⁺OTII⁺ mice (blue) and littermate control HOD^{NEG}OTII+ mice (green) (n=4 per genotype). (B) Dot plot showing pathway enrichment from differential gene expression analysis. Dot size represents the number of genes in each pathway; red to blue scale of p-values indicates red<purple<blue. (C) Plot representing upregulated transcripts in CD4⁺ T cells from autoreactive HOD⁺OTII⁺, compared to littermate control HOD^{NEG}OTII⁺ mice. Transcripts are grouped in colors: those associated with anergy and/or exhaustion (red), immunosuppressive cytokines (green), regulatory T cells (blue), and purinergic signaling (black). Data are combined from 4 mice of each genotype. (D) Representative histograms of checkpoint molecules PD1,Lag-3, and CTLA-4 expression on CD4⁺ T cells from HOD⁺OTII.Rag2p-GFP (black) and littermate control HOD^{NEG}OTII.Rag2p-GFP (grey) mice. CD4⁺ T cells from HOD⁺OTII.Rag2p-GFP (top row) and HOD^{NEG}OTII.Rag2p-GFP (bottom row) mice were sorted based on GFP expression, an indirect measure of T cell age: GFP^{HI} (i.e., RTEs) on the left, GFP^{INT} in the middle and GFP^{LO} (i.e., mature naïve T cells) on the right. Expression of CD73 and CD39 on (E) total CD4⁺Vα2⁺Vβb5⁺ OTII T cells and on (F) CD4⁺FoxP3⁺CD25⁺ Vα2⁺VR5⁺ Tregs. Data in D and E are representative of 3 mice/group and 3 independent experiments.

FIG. 2—ICPi treatment promotes AIHA development in HOD⁺OTII⁺ mice. Young (2-3 months old) male and female HOD⁺OTII⁺ mice were treated 14 days with antibodies against PD1, Lag-3, CTLA-4 and IL-10R (i.e., ICPi, represented in red). Control mice were treated with PBS (black). Whole blood was collected at baseline, after 14 days of treatment, and 14 days post treatment cessation, and (A) sera were analyzed for RBC autoantibodies by flow crossmatch. (B) The frequency of autoantibodies after 14 days of ICPi treatment was stratified by sex: 28/36 females and 18/28 males produced autoantibodies. (C) Hematocrit and bilirubin concentration demonstrates that ICPi-treated (red) but not control (black) mice exhibited RBC hemolysis and became anemic. (D) Linear regression analysis showing a significant negative correlation between RBC autoantibody levels and hematocrit (R²=0.47; P<0.0001). (E) Serum cytokine levels two weeks post ICPi or PBS control treatment. (F) Levels of reactive oxygen species (ROS) in peripheral RBCs. Data in A, B, C, and F are combined from 6 independent experiments with 4 mice/group. Data for bilirubin are combined from 3-5 independent experiment with 4 mice/group. Data in E are combined from 2 independent experiment with 10 mice/group. Statistical significance was determined by Pearson Correlation, Mann-Whitney test, or repeated measures two-way ANOVA with Sidak's multiple comparisons test; p-value *<0.05, ***<0.001, ****<0.0001.

FIG. 3—ICPi accelerates autoantibody production and lupus erythematosus onset in young NZB mice. NZB mice (6-week old females) were treated with ICPi (red) or PBS (black) for 28 days. (A) Autoantibodies bound to peripheral CD41⁺ platelets, CD45⁺ white blood cells, and Ter 19+RBCs was assessed before and during ICPi treatment with a direct anti-globulin test (DAT) by staining with anti-mouse Igs APC. (B) Hematocrit was evaluated before, during (at 14 days) and the cessation of ICPi treatment (at 28 days). Serum was collected to quantify (C) ds-DNA autoantibodies by ELISA at ICPi treatment cessation and (D) BUN and creatinine at the time of euthanasia (i.e., 3 months after treatment cessation). Kidneys from ICPi- and PBS-treated NZB mice were collected, formalin-fixed and paraffin-embedded. Histologic findings in PAS-stained coronal kidney sections were analyzed. (E) Percent of total cortical parenchyma with interstitial inflammation, interstitial fibrosis/tubular atrophy (IFTA) and (F) the number of arteritis lesions per tissue section was calculated. (G) Glomerular features including mesangial hypercellularity, mesangial deposits, endocapillary hypercellularity, leukocyte infiltration, subendothelial and intracapillary deposits, crescents and glomerulosclerosis were graded semiquantitatively on a scale of 0-3+(0=absent; 1+=involving 1-25% glomeruli; 2+=involving 26-50% glomeruli; 3+=involving >50% glomeruli per coronal section of kidney containing over 150 glomeruli per mouse. (H) Representative histological PAS-stained kidney sections are shown. Data are cumulative from 2 independent experiments with 10 mice/group. Statistical significance was determined by Mann-Whitney test or repeated measures two-way ANOVA with Sidak's multiple comparisons test; p-value *<0.05, **<0.01, ****<0.0001.

FIG. 4—ICPi treatment promotes expansion of proinflammatory CD4⁺ T cells. Spleens were collected from ICPi- (red) and PBS-(black) treated young (2-3 months old) HOD⁺OTII⁺ mice after 14 days of treatment. Splenocytes were processed into single cell suspensions and stained with antibodies to delineate T cell subsets. The frequency of (A) regulatory T cells identified as FoxP3⁺ Tregs (CD4⁺CD25⁺FoxP3⁺) and Tr Tregs (CD4⁺FoxP3⁻CD25⁻CD49b⁺Lag3⁺IL10⁺) of total CD4⁺ T cells was calculated. To determine Tr Tregs, enriched CD4 T cells were stimulated for 72 hours at 37 °C with PMA (50 ng/ml) and ionomycin (500 ng/ml), incubated 1 hour with BrefeldinA, and stained to identify the Treg subset. CD4⁺ T cells were stained with antibodies against CD62L and CD44 to quantify the percentage of (B) CD62L⁺CD44⁻ naïve and CD62L⁻CD44⁺ effector cells. To determine T helper cells, enriched CD4 T cells were stimulated for 24 hours at 37 °C with PMA (50 ng/ml) and ionomycin (500 ng/ml), incubated 1 hour with BrefeldinA, and stained intracellularly for IFNy and IL-17A. (C) Representative flow plots and frequency of (D) Th1 and (E) Th17 cells. (F) Ratio of Tregs:Th17 cells was calculated for both groups of mice. (G) Frequency of CD4⁺ T cells expressing CD200, ICOS, PD1, Lag-3, and CTLA-4. Different clones than those contained within the ICPi cocktail were used for PD1, Lag-3, and CTLA-4 staining. Data shown are cumulative of 4-5 independent experiments with 5 mice/group. Statistical significance was determined by Mann-Whitney test; p-value *<0.05, **<0.01, ****<0.0001.

FIG. 5—CD39SP exhibit a proinflammatory T helper phenotype and stimulate B cell proliferation. Spleens from ICPi-(red) and PBS-(black) treated young (2-3 months old) HOD⁺OTII⁺ mice were collected 14 days after treatment and CD4 enriched. (A) Frequency of T cells expressing purinergic signaling molecules CD39 and CD73. (B) Representative flow plots of expression of CD39 and CD73 on total CD4+Vα2⁺Vβ5⁺ OTH T cells (black) and CD4+FoxP3⁺CD25⁺ Vα2⁺VR5⁺ Tregs (red) are shown. (C) Frequency of CD39⁺ CD4⁺ T cells expressing FoxP3. (D) Representative flow plots of expression of CD39 and CD73 on CD4+Vα2⁺Vβ5⁺ OTII T cells (black) and CD49b⁺Lag3⁺IL10⁺ Tr Tregs (red). These data show that CD39⁺ do not express Treg markers and will be defined phenotypically as CD4⁺CD39⁺CD73⁻FoxP3⁻CD25⁻ and referred to as CD39SP. The frequency of CD39SP that stained positive for (E) p-S6 and p-Akt, markers of mTOR complex 1 and 2 activation, (F) HIF1α hypoxia transcription factor, (G), transcription factors c-MAF and T-bet (H) pro-inflammatory cytokines IFNγ and IL-17 was determined. (I) CD39SP were sorted from ICPi- (red) and PBS- (black) treated animals and co-cultured with B cells at 1:2 ratio, previously labeled with CellTrace-FR, and stimulated with HEL or HEL+OVA. CellTrace-FR dilution as measure of B cell proliferation was assessed 3 days after stimulation. CD4⁺ T cells enriched from OTII mice were used as a positive control. Data presented in A-H are cumulative from 4-5 independent experiments with 3-4 mice/group. Data shown in I are cumulative of 2 independent experiments with 4-5 mice/group. Statistical significance was determined by two-way ANOVA with Sidak's post-test and Mann-Whitney test; p-value *<0.05, **<0.01, ***<0.001, ****<0.0001.

FIG. 6—CD39SP precede autoantibody production. Whole blood and sera were collected from young (2-3 months old) HOD⁺OTII⁺ mice before, during (3 and 7 days) and at the cessation of ICPi or PBS treatment (~14 days); (A) representative flow plots and (B) frequency of CD39SP in peripheral whole blood and (C) RBC autoantibodies in sera are shown at each time point. (D) Representative flow plots of FoxP3 and CD25 staining in CD3+CD4+CD45RA− gated T cells isolated from human patients with AIHA (n=9) and healthy volunteers (n=15). Expression of CD39 and CD73 on gated FoxP3⁺ Tregs (red box) and on gated FoxP3⁻CD25⁻ T cells (blue box) were shown. (E) Frequencies of CD39SP with phenotype CD4⁺CD39⁺CD73⁻FoxP3⁻CD25⁻ and (F) FoxP3+ Tregs were detected in PBMCs from ARIA patients (not treated with ICPi) and healthy volunteers. Age-matched healthy volunteers are colored in blue. Data presented in A-C are cumulative from 4-5 independent experiments with 3-4 mice/group. Statistical significance was determined by Mann-Whitney test; p-value *<0.05, **<0.01, ***<0.001, ****<0.0001.

FIG. 7—Prophylactic and therapeutic administration of apyrase ameliorates ICPi-induced AIHA. Whole blood was collected from young (2-3 months old) HOD⁺OTII⁺ mice treated with apyrase or with an inactivated apyrase before every ICPi injection. (A) Sera were analyzed for RBC autoantibodies by flow crossmatch for HOD-specific autoantibodies and (B) hematocrit percentage was calculated. (C) Frequencies of FoxP3⁺ Tregs and (D) CD39SP were calculated. (E) Suppressive activity of CD4⁺CD25⁺ T cells collected from apyrase-pretreated (green), inactive apyrase-pretreated (blue), ICPi-(red) and PBS-treated (black) HOD⁺OTII⁺ mice. Suppression was assessed by adding CD4⁺CD25⁻ T cells (Tconv) in culture with serial dilutions of CD4⁺CD25⁺ enriched T cells. Cells were stimulated with anti-CD3 10 ug/ml and anti-CD28 20 ug/ml for 72 hours at 37 °C. Suppression was expressed as percentage of decreased number of Tconv. (F) Kaplan-Meier survival curve and (G) CD39SP level in whole blood of mice treated with ICPi for 14 days followed by injection of PBS (red line) or 5 units of apyrase daily for 14 days (green line). (H) Linear regression analysis showing a significant positive correlation between RBC autoantibody levels and frequency of CD39SP (R²=0.62; P<0.0001). (I) Young (2-3 months) HOD⁺OTII⁺ mice were treated with ICPi for 14 days alone (red; n=13) or with apyrase treatment beginning at 7 days (i.e., when CD39SP are detectable) (green; n=30). Sera from B6 animals were collected as controls (black; n=3). Sera were serially diluted and analyzed for RBC autoantibodies then analyzed in a flow crossmatch. (J) Hematocrit percentage for mice treated with ICPi alone or with apyrase was calculated. Data presented in A-D are combined from 3 different experiments with 4 mice/group. Data presented in E and F are combined from 2 different experiments. Statistical significance was determined by two-way ANOVA with Sidak's post-test, Mann-Whitney test and by log-rank test; p-value *<0.05, **<0.01, ***<0.001, ****<0.0001

FIG. 8—Schematic showing the role of CD4⁺CD39⁺ T cells in autoimmune hemolytic anemia (AIHA).

FIG. 9—Checkpoint molecule and purinergic signaling molecule expression in CD4⁺ T cells from HODxOTII.Rag2p-GFP F1 mice. Splenic CD4⁺ T cells from HODxOTII.Rag2p-GFP F1 mice were sorted based on GFP expression, an indirect measure of T cell age: GFP^{HI} (i.e., RTEs) on the left, GFP^{INT} in the middle and GFP^{LO} (i.e., mature naïve T cells) on the right. (A) Relative expression of checkpoint molecules ICOS, GITR, CD200 and Tigit. Frequency of (B) FoxP3⁺CD25⁺ Tregs, (C) CD73+, and (D) CD39⁺CD73⁺CD4⁺ T cells. Data shown are representative of 3 mice/group from 3 independent experiments. Statistical significance was determined by two-way ANOVA with Sidak's multiple comparisons test and; p-value *<0.05, **<0.01, ***<0.001, ****<0.0001.

FIG. 10—ICPi treatment leads to anemia and proinflammatory cytokine production. Whole blood collected from young (2-3 months old) male and female HOD⁺OTII⁺ mice treated for 14 days with ICPi (represented in red) was analyzed to assess for anemia. Control mice were treated with PBS (black). After 14 days of ICPi treatment (A) autoantibodies bound to peripheral RBCs was determined with a direct anti-globulin test (DAT) by staining with anti-mouse Igs APC. (B) Hemoglobin and Ter119⁺CD71^{HI} reticulocyte frequency were evaluated at baseline and at 14 days. (C) Quantification of cytokines present in sera of ICPi- and PBS-treated mice. Statistical significance was determined by Mann-Whitney test, or repeated measures two-way ANOVA with Sidak's multiple comparisons test; p-value **<0.01, ***<0.001, ****<0.0001.

FIG. 11—Production of autoantibodies in B6 animals treated with ICPi. Female (3 month old) B6 mice (n=15/group) were treated with ICPi (red) or PBS (black) for 28 days. Autoantibodies bound to peripheral (A) CD41⁺ platelets, CD45⁺ white blood cells, and Ter 119⁺ RBCs was assessed before and during treatment with a direct anti-globulin test (DAT) by staining with anti-mouse Igs APC. (B) Hematocrit, hemoglobin, (C) bilirubin and (D) reticulocytes were evaluated before, during (at 14 days) and at the cessation of ICPi treatment (at 28 days). Serum was collected to quantify (E) ds-DNA autoantibodies by ELISA at ICPi treatment cessation. Statistical significance was determined by repeated measures two-way ANOVA with Sidak's multiple comparisons test; p-value *<0.05, **<0.01, ***<0.001.

FIG. 12—Extrapolated numbers for T cell subsets. Spleens were collected from ICPi- (red) and PBS-treated (black) HOD⁺OTII⁺ mice after 14 days of treatment. Splenocytes were processed into single cell suspensions and stained with antibodies to delineate T cell subsets. The extrapolated cell numbers for (A) CD4⁺ T cells, FoxP3⁺ Tregs (CD4⁺CD25⁺FoxP3⁺), Tr Tregs (CD4⁺FoxP3⁻CD25⁻CD49b⁺Lag3⁺IL10⁺), CD62L⁺CD44⁻ naïve T cells, and CD62L⁻CD44⁺ effector T cells, Th1, and Th17 were calculated. To determine Tr Tregs, enriched CD4⁺ T cells were stimulated for 72 hours at 37 °C with PMA (50 ng/ml) and ionomycin (500 ng/ml), incubated 1 h with BrefeldinA, and stained. To determine T helper cells, enriched CD4 T cells were stimulated for 24 hours at 37 °C with PMA (50 ng/ml) and ionomycin (500 ng/ml), incubated 1 hour with BrefeldinA, and stained for IFNγ and IL-17A. (B) Numbers of CD4⁺ T cells expressing CD200, ICOS, PD1, Lag-3, and CTLA-4. Different clones than those contained within the ICPi cocktail were used for PD1, Lag-3, and CTLA-4 staining. Data shown are cumulative of 4-5 independent experiments with 5 mice/group. Statistical significance was determined by Mann-Whitney test; p-value *<0.05, **<0.01, ***<0.001, ****<0.0001.

FIG. 13—CD39SP are not Tregs and correlate with RBC autoantibody production multiple mouse models. (A) Representative flow plots of CD39 and CD73 staining in CD4⁺FoxP3⁺CD25⁺ Tregs, CD4⁺FoxP3⁻CD25⁻ cells, and total CD4 T cells in ICPi- and PBS-treated animals. (B) Representative flow plots of FoxP3 and CD25 staining in CD4 T cells from ICPi-treated animals. FoxP3⁺CD25⁺ Tregs were further assessed for CD39 and CD73 expression. (C) Representative flow plots of CD39 and CD73 staining in CD4⁺Lag3⁺CD49⁺ Tr cells CD4⁺Lag3⁻CD49⁻ T cells in ICPi-treated animals. (D) Representative flow plots and frequency of expression of CD39 and CD73 on CD4 T cells (black), with CD62L^{high} CD44^{low} naïve T cells, CD62L^{high}CD44^{high} central memory T cells, or CD62L^{low}CD44^{high} effector T cells overlaid in red. The frequency of naïve, central memory, and effector T cells of CD39SP was calculated. (E) Frequency of CD39SP in enriched CD4⁺ T cells collected from old (5-6 months) HOD⁺OTII⁺ mice with (blue; n=15) and without (grey; n=15) detectable RBC autoantibodies. (F) Percent CD39SP detectable in whole blood collected from NZB and (G) B6 mice treated with ICPi (red; n=10) or PBS (black; n=10). (H) Frequency of CD4⁺ T cells expressing PD1, Lag-3, and CTLA-4 in ICPi-treated animals (red; n=10) and apyrase-pretreated animals (green; n=10). Statistical significance was determined by Mann-Whitney test or repeated measures two-way ANOVA with Sidak's post-test; p-value *<0.05, ****<0.0001.

The figures herein are for illustrative purposes only and are not necessarily drawn to scale.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E.A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

The term “optional” or “optionally” means that the subsequent described event, circumstance or substituent may or may not occur, and that the description includes instances where the event or circumstance occurs and instances where it does not.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may in context. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

Immune checkpoint inhibitors (ICPi) have revolutionized cancer immunotherapy but also can induce autoimmune hemolytic anemia (AIHA), a severe disease with high mortality. However, the cellular and molecular mechanism(s) of ICPi-AIHA are unclear, other than being initiated through checkpoint molecule(s) inhibition. Herein, we report ICPi-AIHA in a novel mouse model that shows similar characteristics of known human ICPi-AIHA (e.g., autoantibodies, hemolysis, increased mortality). We also discovered a novel CD39⁺ T cell population that predicts AIHA onset due to ICPi therapy and demonstrate that purinergic signaling modulators, such as apyrase, is a novel therapeutic, effectively mitigating ICPi-AIHA.

During ICPi-AIHA, there is the simultaneous reduction of two regulatory T cell populations (FoxP3⁺ and Tr Tregs) and an increase in inflammatory Th17 T cells. Moreover, a novel CD39⁺CD73⁻FoxP3⁻CD25⁻ CD4⁺ T cell subset (i.e., CD39 single positive [CD39SP]) emerges, and early increases in CD39SP predict AIHA development; CD39 is an ectonuclease that converts extracellular ATP (eATP) and/or ADP into AMP. CD73 then converts AMP into adenosine. Whereas ATP and ADP are proinflammatory molecules, chronically high eATP levels promote inflammation, inhibit FoxP3 expression, and induce conversion of Tregs into Th17 cells, further exacerbating the Th17:Treg imbalance (Yuan et al., Editorial: purinergic signaling and inflammation. Front Immunol 2021, 12:699069), adenosine can be immunosuppressive, promote Treg generation, and prevent T cell activation and proliferation.

As shown in the Examples, boosting ATPase activity by injecting recombinant apyrase mitigates AIHA development and significant CD39SP reductions. Accordingly, targeting CD39 is a novel therapeutic approach for AIHA. Importantly, CD39SP cells are detectable in multiple mouse models developing AIHA and in patients with AIHA, demonstrating applicability to idiopathic and secondary AIHA. Highlighting broader autoimmunity relevance, ICPi-treated NZB mice experienced accelerated onset and severity of lupus, including AIHA. Moreover, ICPi treatment of healthy B6 animals led to detectable CD39SP and development of autoantibodies against multiple autoantigens including those on red blood cells and platelets. Together, our findings elucidate cellular and molecular mechanisms of ICPi-AIHA, leading to novel diagnostic and therapeutic approaches with translational potential for use in humans being treated with ICPi.

### TERMINOLOGY AND DEFINITIONS

In describing implementations for detecting and treating ICPi-induced immune-related adverse events (irAEs), the following terminology will be used in accordance with the definitions and explanations set out below. Notwithstanding, other terminology, definitions, and explanations may be found throughout this document as well.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The term “immune cell” as used throughout this specification generally encompasses any cell derived from a hematopoietic stem cell that plays a role in the immune response. The term is intended to encompass immune cells of the innate and/or adaptive immune system, depending on context. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4⁺, CD8⁺, effector Th, memory Th, regulatory Th, CD4⁺/CD8⁺ thymocytes, CD4-/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

As used throughout this specification, “immune response” refers to a response by a cell of the immune system, such as a B cell, T cell (CD4⁺ or CD8⁺), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”) and refers to a response by a CD4 T cell, CD8 T cell, or B cell via their antigen-specific receptor. In some embodiments, an immune response is a T cell response, such as a CD4⁺ response or a CD8⁺ response. Such responses by these cells can include, for example, cytotoxicity, proliferation, cytokine or chemokine production, trafficking, or phagocytosis, and can be dependent on the nature of the immune cell undergoing the response.

T cell response refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of MHC class I restricted Cytotoxic T lymphocytes (CTLs), may include cytokine and/or cytolytic capabilities, such as lysis of target cells presenting an antigen peptide recognized by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for MHC class II restricted T helper (Th or TH) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific antibody response), antigen presentation, and/or cytokine secretion.

During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8⁺ or CD4⁺ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8⁺ T cell that expresses the CD8⁺ cell surface marker. Such CD8⁺ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8⁺ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8⁺ T cells, may further produce (increased amounts of) one or more immunosuppressive transcription factors or cytokines, such as IL-10 and/or FoxP3, compared to functional immune cells, thereby contributing to local immunosuppression. Dysfunctional CD8⁺ T cells can be both protective and detrimental against disease control. As used herein, a “dysfunctional immune state” refers to an overall suppressive immune state in a subject or microenvironment of the subject (e.g., tumor microenvironment). For example, increased IL-10 production leads to suppression of other immune cells in a population of immune cells.

CD8⁺ T cell function is associated with their cytokine profiles. It has been reported that effector CD8⁺ T cells with the ability to simultaneously produce multiple cytokines (polyfunctional CD8⁺ T cells) are associated with protective immunity in patients with controlled chronic viral infections as well as cancer patients responsive to immune therapy (Spranger et al., 2014, J. Immunother. Cancer, vol. 2, 3). In the presence of persistent antigen CD8⁺ T cells were found to have lost cytolytic activity completely over time (Moskophidis et al., 1993, Nature, vol. 362, 758-761). It was subsequently found that dysfunctional T cells can differentially produce IL-2, TNFa and IFNg in a hierarchical order (Wherry et al., 2003, J. Virol., vol. 77, 4911-4927).

As used herein, terms such as “Th17 cell” and/or “Th17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNy). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses FoxP3.

Depending on the cytokines used for differentiation, in vitro polarized Th17 cells can either cause severe autoimmune responses upon adoptive transfer (‘pathogenic Th17 cells’) or have little or no effect in inducing autoimmune disease (‘non-pathogenic cells’) (Ghoreschi et al., 2010; and Lee et al., 2012 “Induction and molecular signature of pathogenic Th17 cells,” Nature Immunology, vol. 13(10): 991-999). In vitro differentiation of naïve CD4 T cells in the presence of TGF-β1⁺IL-6 induces an IL-17A and IL-10 producing population of Th17 cells, that are generally nonpathogenic, whereas activation of naïve T cells in the presence of IL-1β⁺IL-6⁺IL-23 or TGF-β3⁺IL-6 induces a T cell population that produces IL-17A and IFN-γ, and are potent inducers of autoimmune disease induction (Ghoreschi et al., 2010, Lee et al., 2012).

As used herein, terms such as “pathogenic Th17 cell” and/or “pathogenic Th17 phenotype” and all grammatical variations thereof refer to Th17 cells that, exhibit a distinct pathogenic signature where one or more of the following genes or products of these genes is upregulated in Th17 cells: Cxcl3, Il22, Il3, Ccl4, Gzmb, Lrmp, Ccl5, Casp1, Csf2, Ccl3, Tbx21, Icos, Il7r, Stat4, Lgals3 or Lag3. In certain embodiments, the pathogenic signature is elevated in TGF-β3-induced Th17 cells as compared to TGF-β1-induced Th17 cells. As used herein, terms such as “non-pathogenic Th17 cell” and/or “non-pathogenic Th17 phenotype” and all grammatical variations thereof refer to Th17 cells that exhibit a distinct non-pathogenic signature where one or more of the following genes or products of these genes is up-regulated in Th17 cells: Il6st, Il1rn, lkzf3, Maf, Ahr, Il9 or Il10. In certain embodiments, the non-pathogenic signature is elevated in TGF-β1-induced Th17 cells as compared to TGF-β3-induced Th17 cells. In certain embodiments, when induced in the presence of TGF-β3, Th17 cells express a decreased level of one or more genes selected from IL6st, IL1rn, Ikzf3, Maf, Ahr, IL9 and IL10, as compared to the level of expression in TGF-β3-induced Th17 cells.

As used throughout the present specification, the terms “autoimmune disease” or “autoimmune disorder” are used interchangeably refer to diseases or disorders caused by an immune response against a self-tissue or tissue component (self-antigen) and include a self-antibody response and/or cell-mediated response. The terms encompass organ-specific autoimmune diseases, in which an autoimmune response is directed against a single tissue, as well as non-organ specific autoimmune diseases, in which an autoimmune response is directed against a component present in two or more, several or many organs throughout the body.

Examples of autoimmune diseases include, but are not limited to, acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; autoimmune hemolytic anemia, Behçet's disease; coeliac disease; dermatomyositis; diabetes mellitus type I; Goodpasture's syndrome; Graves' disease; Guillain-Barre syndrome (GBS); Hashimoto's disease; idiopathic thrombocytopenic purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anaemia; polyarteritis nodosa; polymyositis; primary biliary cirrhosis; primary myxedema; psoriasis; rheumatic fever; rheumatoid arthritis; Reiter's syndrome; scleroderma; Sjogren's syndrome; systemic lupus erythematosus; Takayasu's arteritis; temporal arteritis; vitiligo; warm autoimmune hemolytic anemia; or Wegener's granulomatosis.

The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocellular carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, astrocytoma, medulloblastoma, craniopharyngioma, ependymoma, pinealoma, hemangioblastoma, acoustic neuroma, oligodendroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

As used herein, the term “CD39” has its general meaning in the art and refers to the CD39 protein also named as ectonucleoside triphosphate diphosphohydrolase-1 (ENTPD1, ATPDase, CD39, NTPDase-1, SPG64, ectonucleoside triphosphate diphosphohydrolase 1). CD39 is an ectoenzyme that hydrolases ATP/UTP and ADP/UDP to the respective nucleosides such as AMP. Ectonucleoside triphosphate diphosphohydrolase-1 (gene: ENTPD1; protein: NTPDasel) also known as CD39 (Cluster of Differentiation 39), is a typical cell surface enzyme with a catalytic site on the extracellular face. ATP causes a pro-inflammatory environment, whereas degradation of ATP into adenosine by the CD39/CD73 pathway leads to an anti-inflammatory environment. CD39 converts ATP (or ADP) to adenosine monophosphate (AMP), which is converted into adenosine by CD73. A substantial portion of the immune suppressive and anti-inflammatory activity of regulatory T cells (Tregs) is due to the adenosine produced by the CD39/CD73 pathway, insofar as Tregs express CD39 and CD73. Reference sequences include NM_001098175, NM_001164178, NM_001164179, NM_001164181, NM_001164182, NP_001091645, NP_001157650, NP_001157651, NP_001157653, NP_001157654, NM_009848, NM_001304721, NP_001291650, NP_033978.

As used herein, the term “apyrase” has its general meaning in the art and refers to the apyrase protein (ATP-diphosphatase, adenosine diphosphatase, ADPase, ATP diphosphohydrolase), which is a calcium-activated plasma membrane-bound enzyme (magnesium can also activate it) (EC 3.6.1.5) that catalyzes the hydrolysis of ATP to yield AMP and inorganic phosphate. Two isoenzymes are found in commercial preparations from S. tuberosum—one with a higher ratio of substrate selectivity for ATP:ADP (approx. 10) and another with no selectivity (ratio 1). It can also act on ADP and other nucleoside triphosphates and diphosphates.

All gene name symbols refer to the gene as commonly known in the art. The examples described herein that refer to the mouse gene names are to be understood to also encompasses human genes, as well as genes in any other organism (e.g., homologous, orthologous genes). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). Any reference to the gene symbol is a reference made to the entire gene or variants of the gene.

### Methods of Treating Autoimmunity

In example embodiments, a subject suffering from an autoimmune response is treated by decreasing extracellular ATP. In example embodiments, the subject is suffering from an autoimmune disease. In example embodiments, the subject is suffering from cancer and is administered an immunotherapy that increases anti-tumor immunity in the subject. In example embodiments, the immunotherapy leads to irAEs. In example embodiments, inhibition of immune checkpoint proteins leads to irAEs.

### Methods of Decreasing Extracellular ATP

To decrease extracellular ATP, a purinergic signaling modulator is administered to a subject to reduce extracellular ATP levels. In example embodiments, apyrase is administered to a subject. In example embodiments, apyrase is injected into a subject. In example embodiments, a gene therapy vector is used to administer apyrase.

### Regulatory Elements

Recombinant expression vectors may include one or more regulatory elements, which may be selected on the basis of the host cells to be used for expression, that is operably linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). The term “operably linked” as used herein also refers to the functional relationship and position of a promoter sequence relative to a polynucleotide of interest (e.g., a promoter or enhancer is operably linked to a coding sequence if it affects the transcription of that sequence). Typically, an operably linked promoter is contiguous with the sequence of interest. However, enhancers need not be contiguous with the sequence of interest to control its expression. The term “promoter”, as used herein, refers to a nucleic acid fragment that functions to control the transcription of one or more polynucleotides, located upstream of the polynucleotide sequence(s), and which is structurally identified by the presence of a binding site for DNA-dependent RNA polymerase, transcription initiation sites, and any other DNA sequences including, but not limited to, transcription factor binding sites, repressor, and activator protein binding sites, and any other sequences of nucleotides known in the art to act directly or indirectly to regulate the amount of transcription from the promoter. A “tissue-specific” promoter is only active in specific types of differentiated cells or tissues.

In another embodiment, the vector of the invention further comprises expression control sequences including, but not limited to, appropriate transcription sequences (i.e., initiation, termination, promoter, and enhancer), efficient RNA processing signals (e.g., splicing and polyadenylation (polyA) signals), sequences that stabilize cytoplasmic mRNA, sequences that enhance translation efficiency (i.e., Kozak consensus sequence), and sequences that enhance protein stability. A great number of expression control sequences, including promoters which are native, constitutive, inducible, or tissue-specific are known in the art and may be utilized according to the present invention.

In another embodiment, the vector of the invention further comprises a post-transcriptional regulatory region. In a preferred embodiment, the post-transcriptional regulatory region is the Woodchuck Hepatitis Virus post-transcriptional region (WPRE) or functional variants and fragments thereof and the PPT-CTS or functional variants and fragments thereof (see, e.g., Zufferey R, et al., J. Virol. 1999; 73:2886-2892; and Kappes J, et al., WO 2001/044481). In a particular embodiment, the post-transcriptional regulatory region is WPRE. The term “Woodchuck hepatitis virus posttranscriptional regulatory element” or “WPRE”, as used herein, refers to a DNA sequence that, when transcribed, creates a tertiary structure capable of enhancing the expression of a gene (see, e.g., Lee Y, et ah, Exp. Physiol. 2005; 90(1):33-37 and Donello J, et al, J. Virol. 1998; 72(6):5085-5092).

The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif (1990).

Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as tissue cells (e.g., Th17 cells) or other particular cell types. Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Also encompassed by the term “regulatory element” are enhancer elements (e.g., adipose specific enhancers or Woodchuck Hepatitis Virus Posttranscriptional Regulatory Element (WPRE)). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein.

### Vector Selection

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. There are no limitations regarding the type of vector that can be used. The vector can be a cloning vector, suitable for propagation and for obtaining polynucleotides, gene constructs or expression vectors incorporated to several heterologous organisms. Suitable vectors include eukaryotic expression vectors based on viral vectors (e.g., adenoviruses, adeno- associated viruses as well as retroviruses and lentiviruses), as well as non-viral vectors such as plasmids.

In one example embodiment, the vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes. Such vectors are referred to herein as “expression vectors.” Vectors for and that result in expression in a eukaryotic cell can be referred to herein as “eukaryotic expression vectors.” In another example embodiment, the vector integrates the gene into the cell genome or is maintained episomally.

In one example embodiment, by means of an AAV viral vector. The terms “adeno-associated virus”, “AAV virion”, and “AAV particle”, as used interchangeably herein, refer to a virion composed of at least one AAV capsid protein (preferably all capsid proteins of a particular AAV serotype) and an encapsidated polynucleotide AAV genome. If the particle comprises a heterologous polynucleotide flanked by AAV inverted terminal repeats (i.e., a polynucleotide that is not a wild-type AAV genome, e.g., a transgene is delivered to a mammalian cell), it is often referred to as an “AAV vector particle” or “AAV vector”. AAV refers to a virus belonging to the genus dependovirus parvoviridae. The AAV genome is approximately 4.7 kilobases long and consists of single-stranded deoxyribonucleic acid (ssDNA), which can be in either the positive or negative orientation. The genome comprises Inverted Terminal Repeats (ITRs), and two Open Reading Frames (ORFs), at both ends of the DNA strand: rep and cap. The Rep framework is formed by four overlapping genes encoding the Rep proteins required for the AAV life cycle. The cap framework contains overlapping nucleotide sequences of the capsid proteins: VP1, VP2, and VP3, which interact together to form an icosahedral symmetric capsid (see, e.g., Carter B, Adeno-assisted viruses and ado-assisted viruses vectors for genetic drive, Lassic D, et al, eds., “Gene Therapy: Therapeutic Mechanisms and Strategies” (Marcel Dekker, Inc., New York, NY, US, 2000); and Gao G, et al, J.Virol.2004; 78(12):6381-6388). The term “adeno-associated virus ITR” or “AAV ITR” as used herein refers to inverted terminal repeats present at both ends of the DNA strand of the genome of an adeno-associated virus. The ITR sequences are required for efficient proliferation of the AAV genome. Another characteristic of these sequences is their ability to form hairpins. This property contributes to its own priming, which allows synthesis of the second DNA strand independent of the priming enzyme. It has also been shown that ITRs are essential for integration and rescue of wild-type AAV DNA into the host cell genome (i.e., chromosome 19 of humans) and for efficient encapsidation of AAV DNA that binds to the resulting fully assembled, DNase-resistant AAV particles.

The term “AAV vector” as used herein further refers to a vector comprising one or more polynucleotides of interest (or transgenes) flanked by AAV terminal repeats (ITRs). Such AAV vectors can be replicated and packaged as infectious viral particles when present in a host cell that has been transfected with a vector that can encode and express Rep and Cap gene products (i.e., AAV Rep and Cap proteins), and wherein the host cell has been transfected with a vector that encodes and expresses proteins from adenovirus open reading frame E4orf6. When an AAV vector is incorporated into a larger polynucleotide (e.g., a chromosome or another vector, such as a plasmid for cloning or transfection), then the AAV vector is typically referred to as a “protein-vector”. This protein-vector can be “rescued” by replication and encapsidation in the presence of AAV packaging functions and the necessary helper functions provided by E4orf6.

In one example embodiment, gene therapy uses an adeno-associated viral (AAV) vector comprising a recombinant viral genome wherein said recombinant viral genome comprises an expression cassette comprising an adipose tissue-specific transcriptional regulatory region operably linked to a polynucleotide encoding for a target gene in Table 1 or other agent to be delivered, such as a genetic modifying agent or component thereof, (AAV vectors can also be used for any compositions described herein, such as a programable nuclease). AAV according to the present invention can include any serotype of the 42 serotypes of AAV known or an engineered hybrid serotype.

The genome of the AAV according to the invention typically comprises the cis- acting 5′ and 3′ inverted terminal repeat sequences and an expression cassette (see, e.g., Tijsser P, Ed., “Handbook of Parvoviruses” (CRC Press, Boca Raton, FL, US, 1990, pp. 155-168)). The polynucleotide of the invention can comprise ITRs derived from any one of the AAV serotypes.

In another particular embodiment, the AAV vector is a pseudotyped AAV vector (i.e., the vector comprises sequences or components originating from at least two distinct AAV serotypes). In a particular embodiment, the pseudotyped AAV vector comprises an AAV genome derived from one AAV serotype (e.g., AAV2), and a capsid derived at least in part from a distinct AAV serotype. The AAV of the invention comprises a capsid from any serotype.

In one embodiment the AAV vector contains one promoter with the addition of at least one target sequence of at least one miRNA. In some embodiments, the miRNA is endogenous to cells in which it is undesirable to express the polynucleotide cargo of the AAV (e.g., a non-malignant cell). Without being bound by theory, the endogenous miRNA would bind to its target sequence in a transcript produced from the polynucleotide cargo of the AAV and result in degradation of that transcript in the non-target cells (e.g., the non-malignant cells).

In one example embodiment, the vector is a “plasmid,” which refers to a circular double stranded DNA loop into which additional DNA segments can be inserted, such as by standard molecular cloning techniques.

In one example embodiment, the vector is an mRNA vector (see, e.g., Sahin, U, Kariko, K and Tureci, O (2014). mRNA-based therapeutics—developing a new class of drugs. Nat Rev Drug Discov 13: 759-780; Weissman D, Karikó K. mRNA: Fulfilling the Promise of Gene Therapy. Mol Ther. 2015; 23(9):1416-1417. doi:10.1038/mt.2015.138; Kowalski P S, Rudra A, Miao L, Anderson D G. Delivering the Messenger: Advances in Technologies for Therapeutic mRNA Delivery. Mol Ther. 2019; 27(4):710-728. doi:10.1016/j.ymthe.2019.02.012; Magadum A, Kaur K, Zangi L. mRNA-Based Protein Replacement Therapy for the Heart. Mol Ther. 2019; 27(4):785-793. doi:10.1016/j.ymthe.2018.11.018; Reichmuth A M, Oberli M A, Jaklenec A, Langer R, Blankschtein D. mRNA vaccine delivery using lipid nanoparticles Ther Deliv. 2016; 7(5):319-334. doi:10.4155/tde-2016-0006; and Khalil A S, Yu X, Umhoefer J M, et al. Single-dose mRNA therapy via biomaterial-mediated sequestration of overexpressed proteins. Sci Adv. 2020; 6(27):eaba2422). In an exemplary embodiment, mRNA encoding for a target gene is delivered using lipid nanoparticles (see, e.g., Reichmuth, et al., 2016) and administered directly to a tissue. In an exemplary embodiment, mRNA encoding for a target gene is delivered using biomaterial-mediated sequestration (see, e.g., Khalil, et al., 2020) and administered directly to tumor tissue. Sequences present in mRNA molecules, as described further herein, are applicable to mRNA vectors (e.g., Kozak consensus sequence, miRNA target sites and WPRE).

In one example embodiment, the non-viral vector for use in gene transfer and/or nanoparticle formulations is a lipid. In one example embodiment the non-viral lipid vector may comprise: 1,2-Dioleoyl-sn-glycero-3-phosphatidylcholine; 1,2-Dioleoyl-sn-glycero-3-phosphatidylethanolamine; Cholesterol; N-[1-(2,3-Dioleyloxy)propyl]N,N,N-trimethylammonium chloride; 1,2-Dioleoyloxy-3-trimethylammonium-propane; Dioctadecylamidoglycylspermine; N-(3-Aminopropyl)-N,N-dimethyl-2,3-bis(dodecyloxy)-1-propanaminium bromide; Cetyltrimethylammonium bromide; 6-Lauroxyhexyl ornithinate; 1-(2,3-Dioleoyloxypropyl)-2,4,6-trimethylpyridinium; 2,3-Dioleyloxy-N-[2(sperminecarboxamido-ethyl]-N,N-dimethyl-1-propanaminium trifluoroacetate; 1,2-Dioleyl-3-trimethylammonium-propane; N-(2-Hydroxyethyl)-N,N-dimethyl-2,3-bis(tetradecyloxy)-1-propanaminium bromide; Dimyristooxypropyl dimethyl hydroxyethyl ammonium bromide; 3p-[N-(N′,N′-Dimethylaminoethane)-carbamoyl]cholesterol; Bis-guanidium-tren-cholesterol; 1,3-Diodeoxy-2-(6-carboxy-spermyl)-propylamide; Dimethyloctadecylammonium bromide; Dioctadecylamidoglicylspermidin; rac-[(2,3-Dioctadecyloxypropyl)(2-hydroxyethyl)]-dimethylammonium chloride; rac-[2(2,3-Dihexadecyloxypropyl-oxymethyloxy)ethyl]trimethylammonium bromide; Ethyldimyristoylphosphatidylcholine; 1,2-Distearyloxy-N,N-dimethyl-3-aminopropane; 1,2-Dimyristoyl-trimethylammonium propane; O,O′-Dimyristyl-N-lysyl aspartate; 1,2-Distearoyl-sn-glycero-3-ethylphosphocholine; N-Palmitoyl D-erythro-sphingosyl carbamoyl-spermine; N-t-Butyl-NO-tetradecyl-3-tetradecylaminopropionamidine; Octadecenolyoxy[ethyl-2-heptadecenyl-3 hydroxyethyl]imidazolinium chloride; N1-Cholesteryloxycarbonyl-3,7-diazanonane-1,9-diamine; 2-(3-[Bis(3-amino-propyl)-amino]propylamino)-N-ditetradecylcarbamoylme-ethyl-acetamide; 1,2-dilinoleyloxy-3-dimethylaminopropane; 2,2-dilinoleyl-4-dimethylaminoethyl-[1,3]-dioxolane; and dilinoleyl-methyl-4-dimethylaminobutyrate.

In one example embodiment, the non-viral vector for use in gene transfer and/or nanoparticle formulations is a polymer. In one example embodiment the non-viral polymer vector may comprise: Poly(ethylene)glycol; Polyethylenimine; Dithiobis(succinimidylpropionate); Dimethyl-3,3′-dithiobispropionimidate; Poly(ethylene imine) biscarbamate; Poly(L-lysine); Histidine modified PLL; Poly(N-vinylpyrrolidone); Poly(propylenimine); Poly(amidoamine); Poly(amido ethylenimine); Tri ethylenetetramine; Poly(β-aminoester); Poly(4-hydroxy-L-proline ester); Poly(allylamine); Poly(a-[4-aminobutyl]-L-glycolic acid); Poly(D,L-lactic-co-glycolic acid); Poly(N-ethyl-4-vinylpyridinium bromide); Poly(phosphazene)s; Poly(phosphoester)s; Poly(phosphoramidate)s; Poly(N-2-hydroxypropylmethacrylamide); Poly (2-(dimethylamino)ethyl methacrylate); Poly(2-aminoethyl propylene phosphate); Chitosan; Galactosylated chitosan; N-Dodacylated chitosan; Histone; Collagen; and Dextran-spermine.

### Targeted Delivery

In example embodiments, the vector or other polynucleotide for gene knock in can be delivered to the cell using a targeted delivery approach so as to limit delivery and thus expression of a polynucleotide to only cells in which it is delivered. In some embodiments, the delivery vehicle (or composition) used to deliver the vector or other polynucleotide can allow for targeted delivery to a specific cell, tissue, organ, or system. In such embodiments, the delivery vehicle can include one or more targeting moieties that can direct targeted delivery of the cargo(s). In an embodiment, the delivery vehicle comprises a targeting moiety that can target an immune cell. As used herein, “targeting moiety” refers to molecules, complexes, agents, and the like that is capable of specifically or selectively interacting with, binding with, acting on or with, or otherwise associating or recognizing a target molecule, agent, and/or complex that is associated with, part of, coupled to, another object, complex, surface, and the like, such as a cell or cell population, tissue, organ, subcellular locale, particle etc. Targeting moieties can be chemical, biological, metals, polymers, or other agents and molecules with targeting capabilities. Targeting moieties can be amino acids, peptides, polypeptides, nucleic acids, polynucleotides, lipids, sugars, metals, small molecule chemicals, combinations thereof, and the like. Targeting moieties can be antibodies or fragments thereof, aptamers, DNA, RNA such as guide RNA for a RNA guided nuclease or system, ligands, substrates, enzymes, combinations thereof, and the like. The specificity or selectivity of a targeting moiety can be determined by any suitable method or technique that will be appreciated by those of ordinary skill in the art. For example, in some embodiments, the methods described herein include determining the disassociation constant for the targeting moiety and target. In some embodiments, the targeting moiety has a specificity the equilibrium dissociation constant, Kd, is 10⁻³ M or less, 10⁻⁴ M or less, 10⁻⁵ M or less, 10⁻⁶ M or less, 10⁻⁷ M or less, 10⁻⁸M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, the targeting moiety has increased binding with, association with, interaction with, activity on as compared to non-targets, such as a 1 to 500 or more fold increase. Targets of targeting moieties can be amino acids, peptides, polypeptides, nucleic acids, polynucleotides, lipids, sugars, metals, small molecule chemicals, combinations thereof, and the like. Targets can be receptors, biomarkers, transporters, antigens, complexes, combinations thereof, and the like.

### Vector-Based Gene Knock-In

In one example embodiment, the method for treating an autoimmune response by decreasing extracellular ATP comprises administering a recombinant vector encoding apyrase. The vector may further comprise one or more regulatory elements to control expression of the one or more target genes or transcription factors. The vector may further comprise regulatory/control elements, e.g., promoters, enhancers, introns, polyadenylation signals, Kozak consensus sequences, or internal ribosome entry sites (IRES). The vector may further comprise cellular localization signals, such as a nuclear localization signal (NLS) or nuclear export signal (NES). The vector may further comprise a targeting moiety that directs the vector specifically to tumor cells. In another example embodiment, the vector may comprise a viral vector with a tropism specific for immune cells.

### Immunotherapy

In example embodiments, any treatment that decreases ATP as described herein can be administered to a subject receiving an immunotherapy. In example embodiments, the treatment is provided prophylactically, before an immunotherapy is administered. In example embodiments, the treatment is provided at about the same time as an immunotherapy is administered. In example embodiments, the treatment is provided after an immunotherapy is administered. In example embodiments, the treatment is provided before immune-related adverse events. Immunotherapy can include checkpoint blockers (CBP), chimeric antigen receptors (CARs), and adoptive T-cell therapy. As used herein, checkpoint blockade or checkpoint inhibitor therapy refers to a form of cancer immunotherapy. The therapy targets immune checkpoints, key regulators of the immune system that when stimulated can dampen the immune response to an immunologic stimulus. Some cancers can protect themselves from attack by stimulating immune checkpoint targets. Antibodies that block the activity of checkpoint receptors, including CTLA-4, PD-1, Tim-3, Lag-3, and TIGIT, either alone or in combination, have been associated with improved effector CD8⁺ T cell responses in multiple pre-clinical cancer models (Johnston et al., 2014. The immunoreceptor TIGIT regulates antitumor and antiviral CD8(+) T cell effector function. Cancer cell 26, 923-937; Ngiow et al., 2011. Anti-TIM3 antibody promotes T cell IFN-gamma-mediated antitumor immunity and suppresses established tumors. Cancer research 71, 3540-3551; Sakuishi et al., 2010. Targeting Tim-3 and PD-1 pathways to reverse T cell exhaustion and restore anti-tumor immunity. The Journal of experimental medicine 207, 2187-2194; and Woo et al., 2012. Immune inhibitory molecules LAG-3 and PD-1 synergistically regulate T-cell function to promote tumoral immune escape. Cancer research 72, 917-927). Similarly, blockade of CTLA-4 and PD-1 in patients (Brahmer et al., 2012. Safety and activity of anti-PD-L1 antibody in patients with advanced cancer. The New England journal of medicine 366, 2455-2465; Hodi et al., 2010. Improved survival with ipilimumab in patients with metastatic melanoma. The New England journal of medicine 363, 711-723; Schadendorf et al., 2015. Pooled Analysis of Long-Term Survival Data From Phase II and Phase III Trials of Ipilimumab in Unresectable or Metastatic Melanoma. Journal of clinical oncology. official journal of the American Society of Clinical Oncology 33, 1889-1894; Topalian et al., 2012. Safety, activity, and immune correlates of anti-PD-1 antibody in cancer. The New England journal of medicine 366, 2443-2454; and Wolchok et al., 2017. Overall Survival with Combined Nivolumab and Ipilimumab in Advanced Melanoma. The New England journal of medicine 377, 1345-1356) has shown increased frequencies of proliferating T cells, often with specificity for tumor antigens, as well as increased CD8⁺ T cell effector function (Ayers et al., 2017. IFN-gamma-related mRNA profile predicts clinical response to PD-1 blockade. The Journal of clinical investigation 127, 2930-2940; Das et al., 2015. Combination therapy with anti-CTLA-4 and anti-PD-1 leads to distinct immunologic changes in vivo. Journal of immunology 194, 950-959; Gubin et al., 2014. Checkpoint blockade cancer immunotherapy targets tumour-specific mutant antigens. Nature 515, 577-581; Huang et al., 2017. T-cell invigoration to tumour burden ratio associated with anti-PD-1 response. Nature 545, 60-65; Kamphorst et al., 2017. Proliferation of PD-1+ CD8 T cells in peripheral blood after PD-1-targeted therapy in lung cancer patients. Proceedings of the National Academy of Sciences of the United States of America 114, 4993-4998; Kvistborg et al., 2014. Anti-CTLA-4 therapy broadens the melanoma-reactive CD8+ T cell response. Science translational medicine 6, 254ra128; van Rooij et al., 2013. Tumor exome analysis reveals neoantigen-specific T-cell reactivity in an ipilimumab-responsive melanoma. Journal of clinical oncology: official journal of the American Society of Clinical Oncology 31, e439-442; and Yuan et al., 2008. CTLA-4 blockade enhances polyfunctional NY-ESO-1 specific T cell responses in metastatic melanoma patients with clinical benefit. Proceedings of the National Academy of Sciences of the United States of America 105, 20410-20415). Accordingly, the success of checkpoint receptor blockade has been attributed to the binding of blocking antibodies to checkpoint receptors expressed on dysfunctional CD8⁺ T cells and restoring effector function in these cells. The check point blockade therapy may be an inhibitor of any check point protein described herein. The checkpoint blockade therapy may comprise anti-TIM3, anti-CTLA4, anti-PD-L1, anti-PD1, anti-TIGIT, anti-LAG3, or combinations thereof. Anti-PD1 antibodies are disclosed in U.S. Pat. No. 8,735,553. Antibodies to LAG-3 are disclosed in U.S. Pat. No. 9,132,281. Anti-CTLA4 antibodies are disclosed in U.S. Pat. Nos. 9,327,014; 9,320,811; and 9,062,111. Specific check point inhibitors include, but are not limited to anti-CTLA4 antibodies (e.g., Ipilimumab and Tremelimumab), anti-PD-1 antibodies (e.g., Nivolumab, Pembrolizumab, Dostarlimab), and anti-PD-L1 antibodies (e.g., Atezolizumab).

In certain embodiments, immunotherapy leads to immune-related adverse events (irAEs) (see, e.g., Kumar, V. et al. Current Diagnosis and Management of Immune Related Adverse Events (irAEs) Induced by Immune Checkpoint Inhibitor Therapy. Front Pharmacol 8, 49 (2017); Byun et al., (2017) Cancer immunotherapy—immune checkpoint blockade and associated endocrinopathies. Nat Rev Endocrinol. 2017 April; 13(4): 195-207; Abdel-Wahab et al., (2016) Adverse Events Associated with Immune Checkpoint Blockade in Patients with Cancer: A Systematic Review of Case Reports. PLoS ONE 11 (7): e0160221. doi:10.1371/journal.pone.0160221; and Gelao et al., Immune Checkpoint Blockade in Cancer Treatment: A Double-Edged Sword Cross-Targeting the Host as an “Innocent Bystander”, Toxins 2014, 6, 914-933; doi:10.3390/toxins6030914). Thus, patients receiving immunotherapy are at risk for adverse autoimmune responses.

In certain embodiments, irAEs are related to Th17 pathogenicity. In one study, patients treated with ipilimumab had fluctuations in serum IL-17 levels, such that serum IL-17 levels in patients with colitis versus no irAEs demonstrated significantly higher serum IL-17 levels in the patients with colitis (Callahan et al., (2011) Evaluation of serum IL-17 levels during ipilimumab therapy: Correlation with colitis. Journal of Clinical Oncology 29, no. 15_suppl 2505-2505).

In certain embodiments, checkpoint inhibition may be enhanced by administering a TLR agonist to enhance anti-tumor immunity (see, e.g., Urban-Wojciuk, et al., The Role of TLRs in Anti-cancer Immunity and Tumor Rejection, Front Immunol. 2019; 10: 2388; and Kaczanowska et al., TLR agonists: our best frenemy in cancer immunotherapy, J Leukoc Biol. 2013 June; 93(6): 847-863). In certain embodiments, a TLR9 agonist is administered (see, e.g., Chuang, et al., Adjuvant Effect of Toll-Like Receptor 9 Activation on Cancer Immunotherapy Using Checkpoint Blockade, Front. Immunol., 29 May 2020; and Reilley, et al., TLR9 activation cooperates with T cell checkpoint blockade to regress poorly immunogenic melanoma, J. Immunotherapy Cancer, 2019, 7, 323). In certain embodiments, TLR agonists are delivered in a nanoparticle system (see, e.g., Buss and Bhatia, Nanoparticle delivery of immunostimulatory oligonucleotides enhances response to checkpoint inhibitor therapeutics, Proc Natl Acad Sci USA. 2020 Jun. 3; 202001569).

### Administration

A “pharmaceutical composition” refers to a composition that usually contains an excipient, such as a pharmaceutically acceptable carrier that is conventional in the art and that is suitable for administration to cells or to a subject.

As used herein, “carrier” or “excipient” includes any and all solvents, diluents, buffers (such as, e.g., neutral buffered saline or phosphate buffered saline), solubilizers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavorings, aromatizers, thickeners, agents for achieving a depot effect, coatings, antifungal agents, preservatives, stabilizers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

For therapeutic uses, the compositions or agents described herein may be administered systemically, for example, formulated in a pharmaceutically-acceptable buffer such as physiological saline. Preferable routes of administration include, for example, subcutaneous, intravenous, intraperitoneal, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia, although in certain instances lower amounts will be needed because of the increased specificity of the compound.

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

As indicated above, the pharmaceutical compositions according to the invention may be in the form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol.

### Methods of Detecting Autoimmunity

In example embodiments, detection of biomarkers can be used to determine the onset of an irAE in a subject (e.g., CD39, CD73, CD25, FoxP3, IL-17 and/or IFNγ). Biomarkers in the context of the present invention encompasses, without limitation nucleic acids, proteins, reaction products, and metabolites, together with their polymorphisms, mutations, variants, modifications, subunits, fragments, and other analytes or sample-derived measures. The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such. The terms “diagnosis” and “monitoring” are commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

In example embodiments, the genes, biomarkers, and/or cells expressing biomarkers may be detected or isolated by immunofluorescence, immunohistochemistry (IHC), fluorescence activated cell sorting (FACS), mass spectrometry (MS), mass cytometry (CyTOF), sequencing, RNA-seq, single cell RNA-seq, quantitative RT-PCR, single cell qPCR, FISH, RNA-FISH, MERFISH (multiplex (in situ) RNA FISH), Nanostring (see e.g., Geiss G K, et al., Direct multiplexed measurement of gene expression with color-coded probe pairs. Nat Biotechnol. 2008 March; 26(3):317-25), in situ hybridization (ISH), CRISPR-effector system mediated screening assay (e.g., SHERLOCK assay), and any combination thereof. Other methods including absorbance assays and colorimetric assays are known in the art and may be used herein. In certain embodiments, a cancer is diagnosed, prognosed, or monitored. For example, a tissue sample may be obtained and analyzed for specific cell markers (IHC) or specific transcripts (e.g., RNA-FISH). In one embodiment, immune cells are stained for CD39, CD73, CD25, and/or FoxP3. In one embodiment, the cells are fixed. In another embodiment, the cells are formalin fixed and paraffin embedded. Not being bound by a theory, the detection of CD39SP cells with the phenotype CD4⁺CD39⁺CD73⁻FoxP3⁻CD25⁻ indicate outcome and treatments.

The present invention also may comprise a kit with a detection reagent that binds to one or more biomarkers or can be used to detect one or more biomarkers.

### Immunoassays

In example embodiments, immunoassays can be used to determine if subjects are at an increased likelihood of developing an irAE. Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

### Histology

In example embodiments, histology can be used to determine if subjects are at an increased likelihood of developing an irAE. Histology, also known as microscopic anatomy or microanatomy, is the branch of biology which studies the microscopic anatomy of biological tissues. Histology is the microscopic counterpart to gross anatomy, which looks at larger structures visible without a microscope. Although one may divide microscopic anatomy into organology, the study of organs, histology, the study of tissues, and cytology, the study of cells, modern usage places these topics under the field of histology. In medicine, histopathology is the branch of histology that includes the microscopic identification and study of diseased tissue. Biological tissue has little inherent contrast in either the light or electron microscope. Staining is employed to give both contrast to the tissue as well as highlighting particular features of interest. When the stain is used to target a specific chemical component of the tissue (and not the general structure), the term histochemistry is used. Antibodies can be used to specifically visualize proteins, carbohydrates, and lipids. This process is called immunohistochemistry (IHC), or when the stain is a fluorescent molecule, immunofluorescence (IF). This technique has greatly increased the ability to identify categories of cells under a microscope. Other advanced techniques, such as nonradioactive in situ hybridization (ISH), can be combined with immunochemistry to identify specific DNA or RNA molecules with fluorescent probes or tags that can be used for immunofluorescence and enzyme-linked fluorescence amplification.

### Spatial Detection

In example embodiments, spatial detection can be used to determine if subjects are at an increased likelihood of developing an irAE. Methods of generating spatial data of varying resolution are known in the art, for example, ISS (Ke, R. el al. In situ sequencing for RNA analysis in preserved tissue and cells. Nat. Methods 10, 857-860 (2013)), MERFISH (Chen, K. H., Boettiger, A. N., Moffitt, J. R., Wang, S. & Zhuang, X. Spatially resolved, highly multiplexed RNA profiling in single cells. Science 348, (2015)), smFISH (Codeluppi, S. et al. Spatial organization of the somatosensory cortex revealed by cyclic smFISH. biorxiv.org/lookup/doi/10.1101/276097 (2018) doi: 10.1101/276097), osmFISH (Codeluppi, S. et al. Spatial organization of the somatosensory cortex revealed by osmFISH. Nat. Methods 15, 932-935 (2018)), STARMap (Wang, X. et al. Three-dimensional intact-tissue sequencing of single-cell transcriptional states. Science 361, eaat5691 (2018)), Targeted ExSeq (Alon, S. et al. Expansion Sequencing: Spatially Precise In Situ Transcriptomics in Intact Biological Systems. biorxiv.org/lookup/doi/10.1101/2020.05.13.094268 (2020) doi: 10.1101/2020.05.13.094268), seqFISH+(Eng, C.-H. L. et al. Transcriptome-scale super-resolved imaging in tissues by RNA seqFISH+. Nature (2019) doi: 10.1038/s41586-019-1049-y.), Spatial Transcriptomics methods (e.g. Spatial Transcriptomics (ST))(see, e.g., Stahl, P. L. et al. Visualization and analysis of gene expression in tissue sections by spatial transcriptomics. Science 353, 78-82 (2016)) (now available commercially as Visium); Visium Spatial Capture Technology, I OX Genomics, Pleasanton, CA; WO2020047007A2; WO2020123317A2; WO2020047005A1; WO2020176788A1; and WO2020190509A9), Slide-seq (Rodriques, S. G. et al. Slide-seq: A scalable technology for measuring genome-wide expression at high spatial resolution. Science 363, 1463-1467 (2019)), High Definition Spatial Transcriptomics (Vickovic, S. et al. High-definition spatial transcriptomics for in situ tissue profiling. Nat. Methods 16, 987-990 (2019)), or slide-tags (Russell A J C, Weir J A, Nadaf N M, et al. Slide-tags enables single-nucleus barcoding for multimodal spatial genomics [published correction appears in Nature. 2024 January; 625(7994):E11). In certain embodiments, the spatial data can be immunohistochemistry data or immunofluorescence data.

### MS Methods

Biomarker detection may also be evaluated using mass spectrometry (MS) methods. In example embodiments, MS is used to detect biomarkers in non-invasive samples (e.g., blood or stool). A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)2 fragment, a single chain antibody fragment, an Fv fragment, a single chain Fv fragment, a nucleic acid, a lectin, a ligand-binding receptor, affibodies, nanobodies, ankyrins, domain antibodies, alternative antibody scaffolds (e.g., diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

### Single Cell Sequencing

### Sequencing

In example embodiments, sequencing is used to identify expression of genes or transcriptomes in single cells (e.g., RNA-seq). In certain embodiments, sequencing comprises high-throughput (formerly “next-generation”) technologies to generate sequencing reads. Methods for constructing sequencing libraries are known in the art (see, e.g., Head et al., Library construction for next-generation sequencing: Overviews and challenges. Biotechniques. 2014; 56(2): 61-77). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., cDNA) may include sequencing adaptors that are compatible with use in, e.g., Illumina's reversible terminator method, long read nanopore sequencing, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Schneider and Dekker (Nat Biotechnol. 2012 Apr. 10; 30(4):326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncommsl4049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx. doi. org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No.PCT/US2018/060860, published as WO/2019/094984 on May 16, 2019; International Patent Application No. PCT/US2019/055894, published as WO/2020/077236 on Apr. 16, 2020; Drokhlyansky, et al., “The enteric nervous system of the human and mouse colon at a single-cell resolution,” bioRxiv 746743; doi: doi.org/10.1101/746743; and Drokhlyansky E, Smillie C S, Van Wittenberghe N, et al. The Human and Mouse Enteric Nervous System at Single-Cell Resolution. Cell. 2020; 182(6): 1606-1622. e23, which are herein incorporated by reference in their entirety.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25 °C in low stringency wash buffer (IxSSC plus 0.2% SDS) followed by 10 minutes at 25 °C in high stringency wash buffer (0.1 SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif (1992). [0282]Further embodiments are illustrated in the following Examples which are given for illustrative purposes only and are not intended to limit the scope of the invention.

Further embodiments are illustrated in the following Examples which are given for illustrative purposes only and are not intended to limit the scope of the invention.

### EXAMPLES

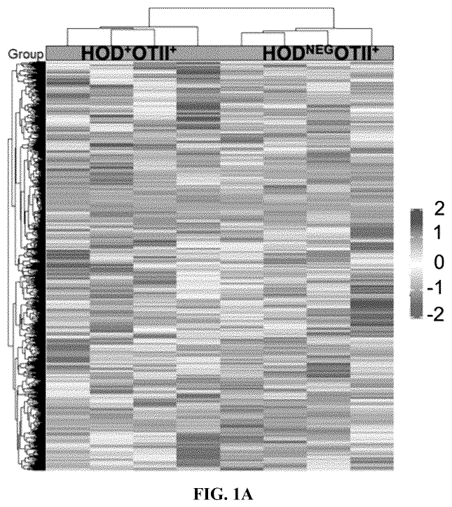

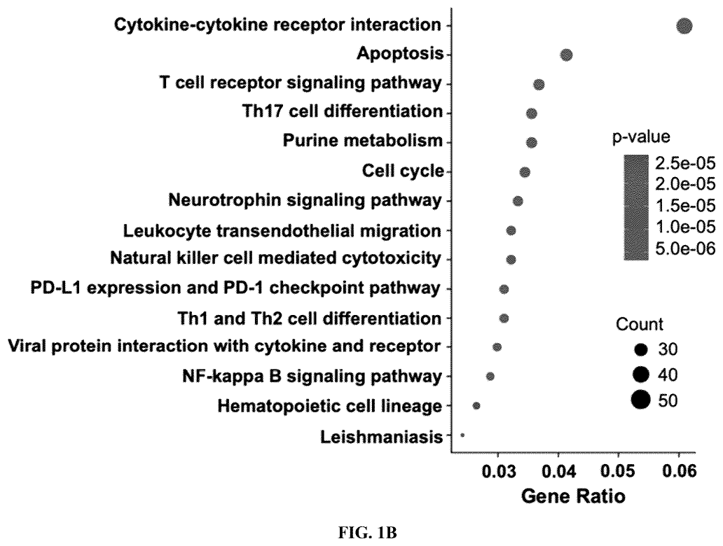

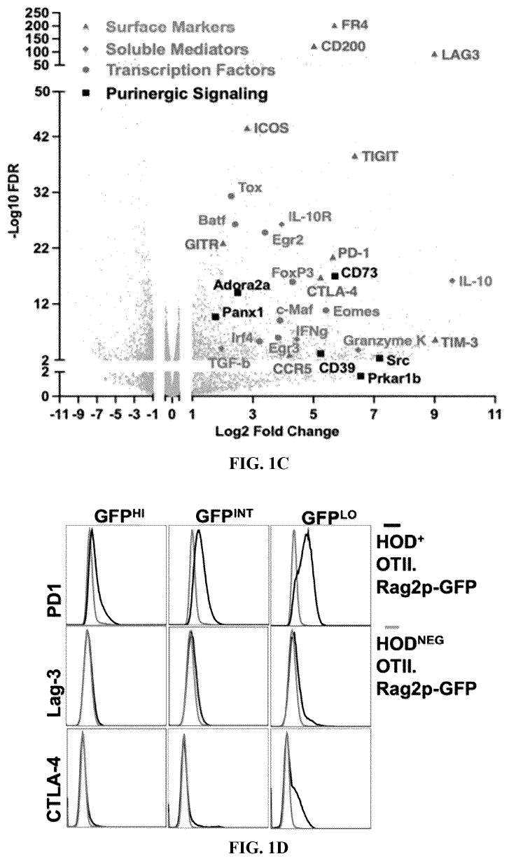

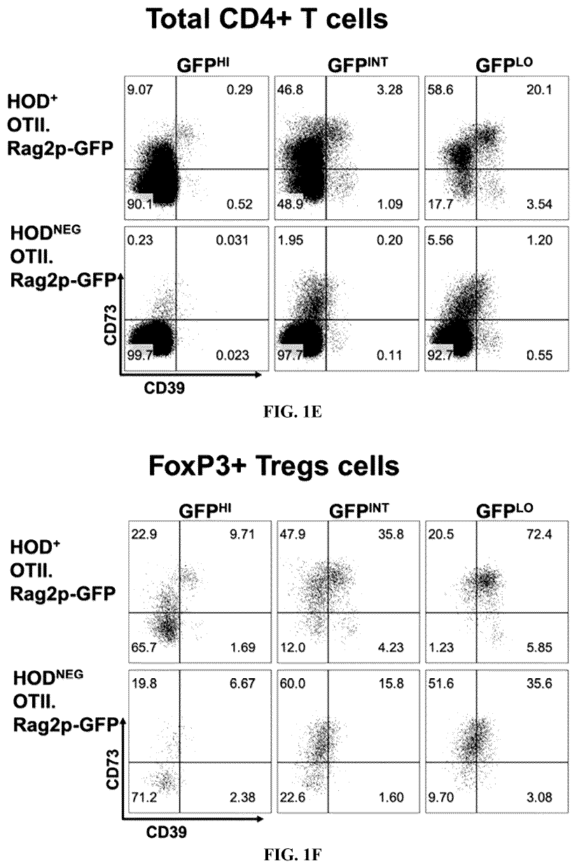

Notwithstanding, with reference to FIGS. 1 to 13 and for the exemplary purposes of this disclosure, many of the implementations relate to the following examples.

### Example 1—Materials and Methods

### Mice.

C57BL/6 (B6) (C57/BL6NCr; #556) mice were purchased from Charles River. OTII (B6.Cg-Tg[TcraTcrb]425CBn/J; #004194) and NZB (NZBWF1/J; #100008) mice were purchased from the Jackson Laboratory. HOD mice and HODxOTII and HODxOTII.Rag2p-GFP F1 mice were generated as described(9, 10, 12). To control for unknown background genetic effects, breeding was performed with heterozygous HOD mice, and HOD⁺OTII⁺ and HOD^{NEG}OTII⁺ mice were littermates. Mice were maintained in a light- and temperature-controlled, pathogen-free environment on standard chow and water, and phenotyped by flow cytometry. Protocols were approved by the Columbia University Institutional Animal Care and Use Committee.

### Human Peripheral Blood Samples.

Anonymized residual whole blood samples from individuals diagnosed with AIHA were collected in EDTA and received from the Columbia University's Center for Advanced Laboratory Medicine through an honest broker protocol approved by the Columbia University Institutional Review Board. Samples were de-identified and only age, sex, and ICD10 codes were collected (Table 1). AIHA diagnosis was established through laboratory evaluation. Peripheral blood mononuclear cells (PBMCs) were isolated as described(13).

TABLE 1
Demographic characteristics, number of CD4⁺ T cells in 1 mL of blood collected from patients with AIHA and healthy volunteers.
Frequency of CD39SP cells with phenotype CD4⁺CD39⁺CD73⁻FoxP3⁻CD25⁻ was calculated. Laboratory evaluation of AIHA patients.

| | | | Number | % of | | | | | | |
|---|---|---|---|---|---|---|---|---|---|---|
| | | | of | FoxP3− | | | | | | |
| Sample | | | CD4+/ | CD39+/ | | Indirect | | | | |
| ID | Sex | Age | mL | CD4+ | Hgb | bilirubin | LDH | Haptoglobin | History | Treatment |
| AIHA-1 | F | 82 | 8,364 | 8.09 | 3.9 | 7.7 | 1396 | <20 | CLL, severe AIHA | High dose steroids, |
| | | | | | | | | | | IVIG, rituximab |
| AIHA-2 | M | 67 | 16,202 | 3.6 | 6.8 | 2.2 | 352 | <20 | systemic | High dose steroids, |
| | | | | | | | | | mastocytosis, | IVIG, rituximab |
| | | | | | | | | | cytopenias, Evans | |
| | | | | | | | | | syndrome | |
| AIHA-3 | M | 59 | 16,855 | 5.29 | 3.9 | 3.9 | 2801 | <10 | Diabetes type2, | High dose steroids, |
| | | | | | | | | | severe AIHA mixed | plasma exchange, |
| | | | | | | | | | warm and cold | rituximab |
| | | | | | | | | | autoantibodies | |
| AIHA-4 | M | 76 | 3,141 | 31.5 | 6.6 | 2.3 | 350 | <20 | Follicular lymphoma, | rituximab |
| | | | | | | | | | hemolysis and warm | |
| | | | | | | | | | autoantibodies | |
| AIHA-5 | M | 71 | 11,056 | 10.4 | 3.1 | 2.7 | 953 | <20 | CLL, AIHA | High dose steroids |
| AIHA-6 | M | 58 | 5,123 | 30.4 | 5.8 | 0.7 | 665 | <20 | AITL, AIHA | High dose steroids |
| | | | | | | | | | | and etoposide, |
| | | | | | | | | | | belinostat for AITL |
| AIHA-7 | M | 4 | 14,612 | 12.6 | 5.2 | 1 | 710 | | Dilated | High dose steroids, |
| | | | | | | | | | cardiomyopathy, pure | rituximab |
| | | | | | | | | | red cell aplasia, AIHA | |
| AIHA-8 | M | 84 | 9,736 | 10.11 | 8.3 | 1.8 | 669 | <20 | CLL, warm and cold | High dose steroids |
| | | | | | | | | | autoantibodies, | |
| | | | | | | | | | severe anemia | |
| AIHA-9 | M | 21 | 9,805 | 7.15 | 6.3 | 2.1 | 403 | <20 | Evans syndrome, | High dose steroids, |
| | | | | | | | | | warm AIHA, CLL | IVIG, rituximab, |
| | | | | | | | | | | cytoxan |
| Healthy-1 | M | 37 | 46,116 | 0.21 | | | | | | |
| Healthy-2 | F | 36 | 46,202 | 3.91 | | | | | | |
| Healthy-3 | F | 23 | 42,875 | 2.3 | | | | | | |
| Healthy-4 | F | 27 | 24,138 | 1.59 | | | | | | |
| Healthy-5 | F | 24 | 45,431 | 2.14 | | | | | | |
| Healthy-6 | F | 42 | 63,190 | 1.45 | | | | | | |
| Healthy-7 | F | 62 | 66,387 | 3.56 | | | | | | |
| Healthy-8 | F | 71 | 47,738 | 4.3 | | | | | | |
| Healthy-9 | M | 71 | 52,723 | 0.9 | | | | | | |
| Healthy-10 | M | 55 | 70,446 | 14.9 | | | | | | |
| Healthy-11 | M | 51 | 110,017 | 3.4 | | | | | | |
| Healthy-12 | F | 65 | 111,893 | 13 | | | | | | |
| Healthy-13 | M | 63 | 63,711 | 3.03 | | | | | | |
| Healthy-14 | M | 50 | 75,011 | 3.65 | | | | | | |
| Healthy-15 | M | 63 | 57,511 | 7.81 | | | | | | |

Hgb: Hemoglobin; LDH: Lactate Dehydrogenase; CLL: Chronic Lymphocytic Leukemia; AITL: Angioimmunoblastic T cell Lymphoma; IVIG: Intravenous Immunoglobulin.

### RNA Sequencing.

Total RNA was extracted from sorted splenic CD4⁺ T cells collected from HODxOTII F 1 mice and sent to the Genome Technology Access Center at Washington University for RNA sequencing. Raw data and additional information on library construction, data processing, and data analysis can be found at datadryad.org/stash/share/hmEl0cX5fC0I-SlgK2fMTy0mYi0PjUJFnnSPMdpAqUY.

### ICPi Cocktail and Apyrase Injections.

Mice were injected intraperitoneally with a 4-antibody cocktail against IL-IOR, CTLA-4, Lag-3 and PD 1. Apyrase from Pichia pastoris was administered for prophylactic (3 units of apyrase or inactivated apyrase(14)) or therapeutic administration (5 units).

### Peripheral Blood Analysis.

Peripheral blood was assessed for autoantibodies using direct anti-globulin test and flow crossmatch, as described(10). To evaluate anemia, Ter119⁺CD71^{high} reticulocyte frequency, hematocrit and hemoglobin were determined as described(15). Reactive oxygen species (ROS) were detected using CM-H₂DCFDA (DCFDA) probe and CD39SP was detected in whole blood. Serum blood urea nitrogen (BUN) and creatinine were measured by the CLIA-certified clinical diagnostic lab at Columbia University (Roche Cobas). Serum ds-DNA autoantibodies, bilirubin and cytokines were quantified using assay kits, per manufacturer's instructions.

### Kidney Pathology.

Kidneys harvested from 6-month-old NZB mice were bivalved and fixed in 10% paraformaldehyde for 24 hours. Periodic acid-Schiff (PAS) staining was performed on 3-micron paraffin embedded tissue sections. Histologic features of lupus nephritis in glomerular, tubulointerstitial and vascular compartments were analyzed and scored by a blind assessor using an Olympus BX41 light microscope.

### Spleen Analysis and Flow Cytometry.

Spleens were processed into single splenocyte suspensions. CD4⁺ and CD39SP T cells were sorted, as described(16), with a Sony MA900 and co-cultured with B cells or stained with antibodies to detect checkpoint molecules, Tregs, purinergic signaling, and naïve, effector and memory T cells. To assess T helper cells (THs) and Tr Tregs, cells were incubated at 37 °C with 50 ng/mL PMA and 500 ng/mL ionomycin for 24- or 72-hours, respectively. In vitro suppression assays were performed using a modified protocol previously described(17).

### Statistical Analysis.

Comparison of means between two groups was analyzed with Mann-Whitney test. Experiments with 3 or more groups were analyzed with a 1-way ANOVA with Sidak's comparisons post-test. For longitudinal analysis of 3 or more groups, a repeated measures 2-way ANOVA with multiple comparisons test was utilized. Data were analyzed in GraphPad Prism and p<0.05 was considered significant.

### Human Peripheral Blood Samples.

Comprehensive laboratory evaluation to diagnose AIHA included hemoglobin level, indirect bilirubin, lactate dehydrogenase, haptoglobin, pan-positive antibody screening and antibody panels, positive autocontrol, and a positive direct antiglobulin test (DAT). In cases of warm AIHA (wAIHA), the DAT exhibited positivity with polyspecific reagent and IgG (with or without complement C3).

Isolated PBMCs from whole blood by Ficoll density gradient were stained for surface markers for 30 minutes at 4 °C with antibodies specific for CD4, CD3, CD45RA, CD39 CD73 and CD25. Intracellular expression of transcription markers for FoxP3 was determined with the FoxP3 transcription factor staining buffer set per manufacturer's instructions. Samples were acquired using an Attune NxT flow cytometer (ThermoFisher) and analyzed with FlowJo software (BD Biosciences).

### RNAseq Analysis.

Differentially expressed genes (rate adjusted p<0.05 and >1.5 fold change) between CD4⁺ T cells from HOD⁺OTII⁺ and HOD^{NEG}OTII⁺ mice were plotted as −log 10(p-value) by log 2 fold change in GraphPad Prism version X. Kobas was used to perform pathway analysis (Xie C, Mao X, Huang J, Ding Y, Wu J, Dong S, et al. KOBAS 2.0: a web server for annotation and identification of enriched pathways and diseases. Nucleic Acids Res. 2011; 39(Web Server issue):W316-22).

### ICPi Cocktail and Apyrase Injections.

B6, NZB and HODxOTII F1 mice were injected intraperitoneally for 14-28 days with a 4-antibody cocktail against IL-10R (500 ug weekly), and CTLA-4, Lag-3 and PD1, (200ug each, every other day). Control mice received PBS. Apyrase from Pichia pastoris was reconstituted at 100 units/mL in sterile PBS; inactivation was achieved by heating at 95 °C for 10 minutes (Coover R A, Healy T E, Guo L, Chaney K E, Hennigan R F, Thomson C S, et al. Tonic ATP-mediated growth suppression in peripheral nerve glia requires arrestin-PP2 and is evaded in NF l. Acta Neuropathol Commun. 2018; 6(1):127). For prophylactic administration, 3 units of apyrase or inactivated apyrase were injected intraperitoneally 1 hour before each ICPi injection. For therapeutic administration, 5 units were infused daily for 10 days, either after 2 weeks or during the ICPi administration but after the appearance of CD39SP in peripheral whole blood.

### Peripheral Blood Analysis.

Peripheral blood was drawn by retro-orbital puncture into heparinized capillary tubes, washed 3 times with FACS buffer, and stained as follows. Autoantibodies bound to Ter119⁺ RBCs, CD41⁺ platelets, and CD45⁺ white blood cells were determined by calculating the mean fluorescence intensity (MFI) of goat anti-mouse Ig APC. CM-H₂DFCDA (DCFDA) staining at 1 μM for 25 minutes at 37 °C was performed to detect reactive oxygen species (ROS). To detect CD39SP, whole blood was hemolyzed, washed, then stained with CD4, Thy1.2, CD39 and FoxP3 antibodies with the FoxP3 transcription factor staining buffer set. Sera was collected from whole blood by centrifugation for 10 minutes at 13,000 rpm. Samples were acquired using an Attune NxT flow cytometer and analyzed with FlowJo.

Spleen analysis and flow cytometry.

CD4⁺ or CD4⁺CD25⁺ T cells were enriched using EasySep Mouse CD4⁺ T cell or CD4⁺CD25⁺ Regulatory T cell Isolation Kits. Enriched CD4⁺ T cells from HODxOTII.Rag2p-GFP Fl mice were sorted based on GFP expression and stain with antibodies against PD1, CTLA-4, Lag-3, CD200, ICOS to detect checkpoint molecules, CD25 and CD49b to assess Tregs, CD39 and CD73 for purinergic signaling, and CD62L and CD44 to determine naïve, effector and memory T cells. Intracellular expression of transcription markers for FoxP3, HIF1α, mTORC1 (pS6), mTORC2 (pAkt) and T helper cells (t-bet, c-Maf, IFNγ and IL17A) were determined with the FoxP3 transcription factor staining buffer set per manufacturer's instructions. To assess T helper cells (THs) and Tr Tregs, cells were permeabilized and stained with IFN-γ, IL-17, or IL-10 antibodies after incubation with 50 ng/mL PMA and 500 ng/mL ionomycin for 24- or 72-hours, respectively. Cytokines were quantified using Legendplex Multi-analyte flow assay kit. Cell analysis was performed using an Attune NxT flow cytometer and analyzed with FlowJo.

### T-B Cell Co-Culture Assay.

Sorted CD39SP isolated from PBS- and ICPi-treated animals were co-cultured with HEL-specific transgenic B cells, previously labeled with 1 μM of cell proliferation dye (CellTrace-FarRed), at a ratio of 1:2 in presence of soluble HEL (1 μl·10 mg/ml) and HEL+OVA (1 μl·1 mg/ml). B cells cultured alone served as a negative control, whereas OTH CD4+ T cells co-cultured with B cells was a positive control. Cocultured cells were harvested 3 days after stimulation, and CellTrace-FarRed dilution was assessed in CD3⁻CD4⁻CD19⁺ B cells.

### T Cell Suppression Assay.

Enriched splenic CD4⁺CD25⁺ T cells from PBS-, ICPi-, and apyrase-treated animals were co-cultured with CD4⁺CD25⁻ conventional T cells at ratio of 1:4, with serial dilutions, on plate-immobilized anti-CD3/CD28 antibodies for 3 days at 37 °C. Proliferation of conventional T cells was determined by flow cytometry and the percent suppression calculated.

Antibodies and cell processing reagents are provided in Table 2.

TABLE 2
List of antibodies and reagents used in studies.

| Antibody | Clone | Fluorophore | Species | Dilution | Source | Reference |
|---|---|---|---|---|---|---|
| anti-human CD4 | RPA-T4 | eF506 | Mouse | 1:100 | eBioscience | 69-0049-42 |
| | | | monoclonal | | | |
| anti-human CD3 | SK7 | PercP-710 | Mouse | 1:100 | Invitrogen | 46-0036-42 |
| | | | monoclonal | | | |
| anti-human | HI100 | APC-eF780 | Mouse | 1:100 | Invitrogen | 47-0458-42 |
| CD45RA | | | monoclonal | | | |
| anti-human CD39 | A1 | FITC | Mouse | 1:200 | Biolegend | 328206 |
| | | | monoclonal | | | |
| anti-human CD73 | AD2 | APC | Mouse | 1:200 | Biolegend | 344006 |
| | | | monoclonal | | | |
| anti-mouse Igs | | APC | Goat polyclonal | 1:100 | Southern | 1012-11 |
| | | | | | Biotech | |
| anti-mouse Igs | | PE | Goat polyclonal | 1:100 | Southern | 1010-09 |
| | | | | | Biotech | |
| anti-mouse Ter119 | TER-119 | BV510 | Mouse | 1:100 | Biolegend | 116237 |
| | | | monoclonal | | | |
| anti-mouse Ter119 | TER-119 | PercP-710 | Rat monoclonal | 1:100 | Invitrogen | 46-5921-82 |
| anti-mouse CD71 | R17217 | FITC | Rat monoclonal | 1:100 | Invitrogen | 11-0711-81 |
| anti-mouse CD41 | MWReg30 | PE | Rat monoclonal | 1:100 | Invitrogen | 12-0411-83 |
| anti-mouse CD45 | 30-F11 | AF700 | Rat monoclonal | 1:100 | Invitrogen | 56-0451-82 |
| anti-mouse CD4 | GK1.5 | BV605 | Rat monoclonal | 1:100 | BD bioscience | 743156 |
| anti-mouse Thy1.2 | 53-2.1 | eF450 | Rat monoclonal | 1:1000 | Invitrogen | 48-0902-82 |
| anti-mouse CD39 | 24DMS1 | PE-Cy7 | Rat monoclonal | 1:200 | Invitrogen | 25-0391-82 |
| anti-mouse CD73 | eBioTY/11.8 | PercP-710 | Rat monoclonal | 1:200 | Invitrogen | 46-0731-82 |
| anti-mouse PD1 | RMP1-30 | FITC | Rat monoclonal | 1:100 | Invitrogen | 11-9981-82 |
| anti-mouse PD1 | J43 | FITC | Rat monoclonal | 1:100 | Invitrogen | 11-9985-82 |
| anti-mouse CTLA-4 | UC10- | APC | Rat monoclonal | 1:100 | Biolegend | 135209 |
| | 4F10-11 | | | | | |
| anti-mouse CTLA-4 | UC10-4B9 | APC | Rat monoclonal | 1:100 | Invitrogen | 17-1522-80 |
| anti-mouse Lag-3 | C9B7W | PE-Cy7 | Rat monoclonal | 1:200 | eBioscence | 25-2231-82 |
| anti-mouse Lag-3 | 11C3C65 | BV510 | Mouse | 1:200 | Biolegend | 369318 |
| | | | monoclonal | | | |
| anti-mouse CD200 | OX90 | PE | Rat monoclonal | 1:100 | eBioscence | 12-5200-82 |
| anti-mouse ICOS | 15F9 | PE | Syrian hamster | 1:100 | Biolegend | 107705 |
| | | | monoclonal | | | |
| anti-mouse CD25 | PC61.5 | APC-eF780 | Rat monoclonal | 1:100 | eBioscience | 47-0251-82 |
| anti-mouse CD49b | HMa2 | FITC | Armenian | 1:100 | Invitrogen | 11-0491-82 |
| | | | hamster | | | |
| | | | monoclonal | | | |
| anti-mouse FoxP3 | FJK-16s | APC | Rat monoclonal | 1:100 | Invitrogen | 17-5773-82 |
| anti-mouse CD62L | MEL-14 | PercP-Cy5.5 | Rat monoclonal | 1:100 | Invitrogen | 45-0621-82 |
| anti-mouse CD44 | IM7 | BV510 | Rat monoclonal | 1:100 | Biolegend | 103044 |
| anti-mouse HIF1α | | APC | Mouse | 1:100 | R&D | IC1935A |
| | | | monoclonal | | | |
| anti-mouse p-S6 | D57.2.2E | AF488 | Rabbit | 1:50 | Cell signaling | 4803S |
| (S235-236) | | | monoclonal | | | |
| anti-mouse | SDRNR | PE | Mouse | 1:50 | Invitrogen | 12-9715-42 |
| phospho-Akt1 | | | monoclonal | | | |
| (Ser473) | | | | | | |
| anti-mouse t-bet | 4B10 | PE-Cy7 | Mouse | 1:100 | Invitrogen | 25-5825-82 |
| | | | monoclonal | | | |
| anti-mouse c-Maf | sym0F1 | PE | Mouse | 1:100 | Invitrogen | 12-9855-42 |
| | | | monoclonal | | | |
| anti-mouse IFNγ | XMG1.2 | APC | Rat monoclonal | 1:50 | Invitrogen | 17-7311-82 |
| anti-mouse IL17A | eBio17B7 | FITC | Rat monoclonal | 1:50 | Invitrogen | 53-7177-81 |
| anti-mouse IL10 | JES5-16E3 | PE | Rat monoclonal | 1:100 | Invitrogen | 12-7101-82 |
| CD28 Monoclonal | 37.51 | | Syrian hamster monoclonal | | eBioscience | 16-0281-86 |
| Antibody | | | | | | |
| Functional Grade | | | | | | |
| CD3e Monoclonal | 145-2C11 | | Armenian hamster | | eBioscience | 16-0031-86 |
| Antibody | | | monoclonal | | | |
| Functional Grade | | | | | | |
| ICPi cocktail and | | | | | | |
| Apyrase | Clone | | | | Source | Reference |
| InVivo Plus anti- | UC10- | | | | BioxCell | BP0032 |
| mouse CTLA-4 | 4F10-11 | | | | | |
| (CD152) | | | | | | |
| InVivoPlus anti- | C9B7W | | | | BioxCell | BP0174 |
| mouse LAG-3 | | | | | | |
| InVivo Plus anti- | RMP1-14 | | | | BioxCell | BP0146 |
| mouse PD-1 | | | | | | |
| (CD279) | | | | | | |
| InVivoPlus anti- | 1B1.3A | | | | BioxCell | BP0050 |
| mouse IL-10R | | | | | | |
| (CD210) | | | | | | |
| Apyrase | | | | | Sigma | A6535 |
| Kit and reagent | | | | | Source | Reference |
| EasySep ™ Mouse CD4+ T Cell Isolation Kit | | | | | StemCellTechnologies | 19852A |
| EasySep ™ Mouse CD4+CD25+ Regulatory T Cell Isolation Kit II | | | | | StemCellTechnologies | 18783 |
| Foxp3/Transcription Factor Staining Buffer Set | | | | | eBioscience | 00-5523-00 |
| CM-H2DFCDA | | | | | Invitrogen | C6827 |
| Bilirubin Assay Kit | | | | | Sigma | MAK126- |
| | | | | | | 1KT |
| Drabkin's reagent | | | | | Ricca Chemical | 2660-32 |

| mouse anti-dsDNA IgG-specific ELISA kit 96 test | alpha- | 5120 |
|---|---|---|
| Ionomycin, Calcium Salt | Diagnostic | |
| Phorbol 12-myristate 13-acetate | Invitrogen | I24222 |
| (PMA) | Sigma | P8139 |
| Brefeldin A (GolgiPlug ™) | | |
| LEGENDplex ™ Mouse Inflammation Panel (13- | BD bioscience | 555028 |
| plex) | Biolegend | 740446 |
| CellTrace ™ Far Red Cell | | |
| Proliferation Kit | Invitrogen | C34564A |

### Example 2—RBC-Autoreactive CD4⁺ T Cells Tolerization Involves Checkpoint Molecules, Treg Development, and Purinergic Signaling Molecules

The HOD mouse expresses an RBC-specific surface protein containing the peptide (OVA₃₂₃₋₃₃₉) recognized by OTII CD4⁺ T cells. We previously reported that RBC-autoreactive CD4⁺ T cells from HOD⁺OTII⁺ are tolerized as RTEs(9), and that tolerance fails, leading to AIHA, upon aging (i.e., ~5 months)(10). To identify RTE tolerization pathways, RNAseq was performed on sorted CD4⁺ T cells from young autoreactive HOD⁺OTII+ and littermate negative control HOD^{NEG}OTII⁺ mice. Hierarchical clustering of transcripts revealed significant differences between tolerized and control CD4⁺ T cells (FIG. 1A). Pathway analysis highlighted significant changes in gene expression in multiple pathways including T cell receptor signaling, Th17 cell differentiation, and purine metabolism (FIG. 1B). Tolerized CD4⁺ T cells from autoreactive HOD⁺OTII+ mice significantly upregulated checkpoint molecules (e.g., PD1, Lag-3, CTLA-4)(18), immunosuppressive cytokines (e.g., IL-10, TGF-P, IL-10R)(19, 20), purinergic signaling molecules (e.g., CD39, CD73)(21), and Treg-associated transcription factors (e.g., FoxP3, Egr2, Eomes) (FIG. 1C). This enrichment of transcription factors and soluble mediators was consistent with at least two known Treg subsets: FoxP3⁺ Tregs that secrete immunosuppressive IL-10 and TGF-P, and Tr Tregs that can express Eomes, c-MAF, Egr2, and IRF4 and secrete granzyme K and IFNγ(22-24).

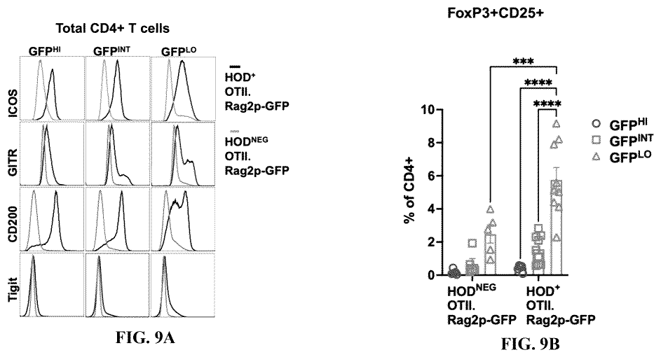

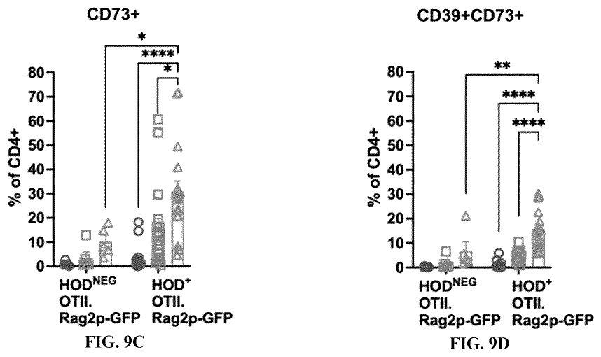

To validate the RNAseq data and evaluate protein expression as RTEs become tolerized, CD4⁺ T cells from HODxOTII.Rag2p-GFP F1 mice were sorted based on GFP intensity, an indirect measure of time since thymic emigration; GFP signal is brightest in RTEs (i.e., “GFP^{HI}”) and decays over 3 weeks as T cells mature (i.e., “GFP^{LO}”)(9). Compared to controls, tolerized CD4⁺ RTEs from HOD⁺OTII.Rag2p-GFP mice progressively upregulated PD1, Lag-3, CTLA-4, ICOS, GITR, and CD200 as they matured (FIG. 1D and FIG. 9A). Consistent with prior observations(9), low FoxP3⁺ Treg frequencies were detectable in autoreactive GFP^{HI} RTEs, which expanded upon maturation (FIG. 1E and FIG. 9B). Based on our RNAseq data and ATP hydrolysis as a well-described Treg suppression mechanism(20, 25), expression of purinergic signaling molecules CD39 and CD73 was interrogated. Compared to controls, there was a significant increase in the frequency of CD73⁺ cells in autoreactive GFP^{HI} RTEs, which further increased during maturation (FIG. 1E and FIG. 9C). Significant frequencies of autoreactive GFP^{INT} and GFP^{LO} RTEs also exhibited detectable co-expression of CD39 and CD73, as compared to controls. FoxP3⁺ Tregs co-expressed CD39 and CD73 (FIG. 1F and FIG. 9D). Intriguingly, CD73 upregulation preceded FoxP3⁺ Treg development, suggesting a role for purinergic signaling in tolerization to RBC autoantigens.

### Example 3—ICPi Reverse Tolerance to RBC Autoantigens and Initiate AIHA

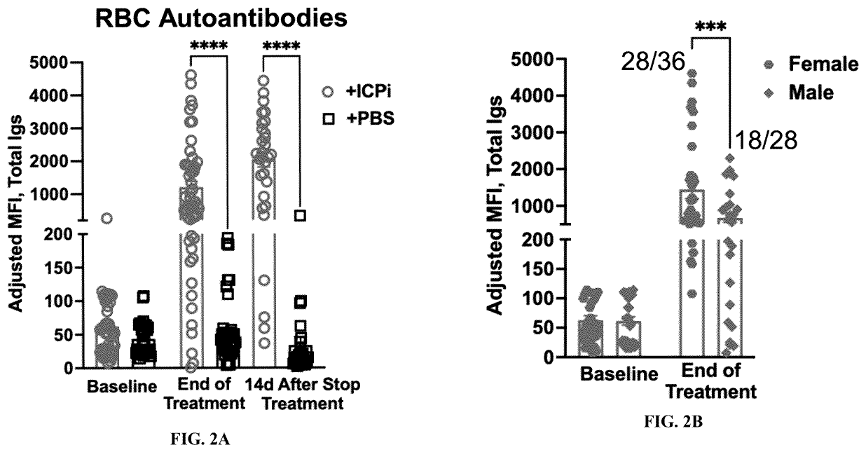

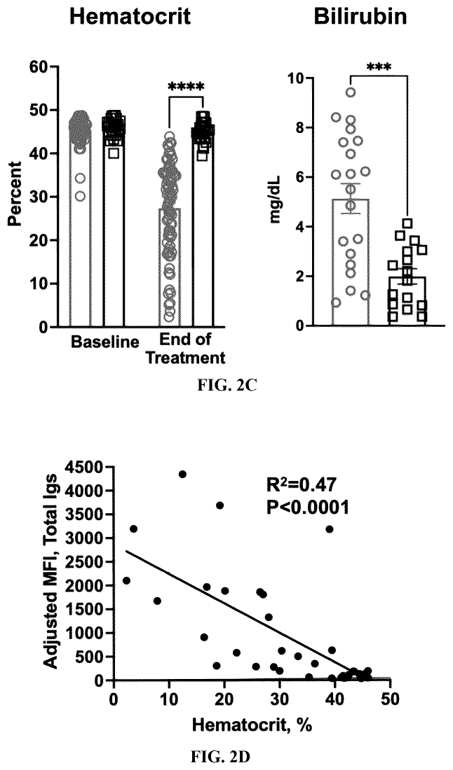

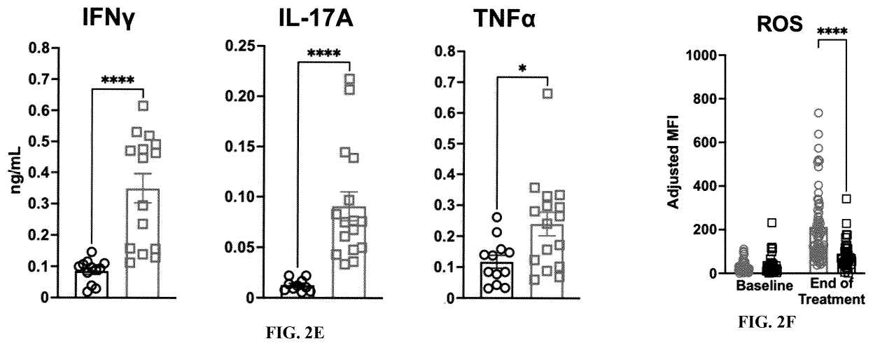

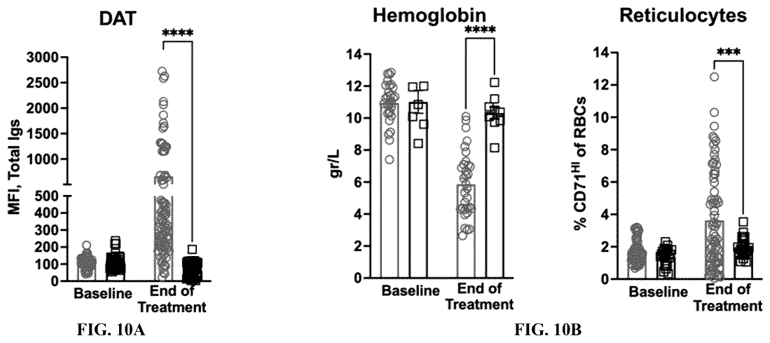

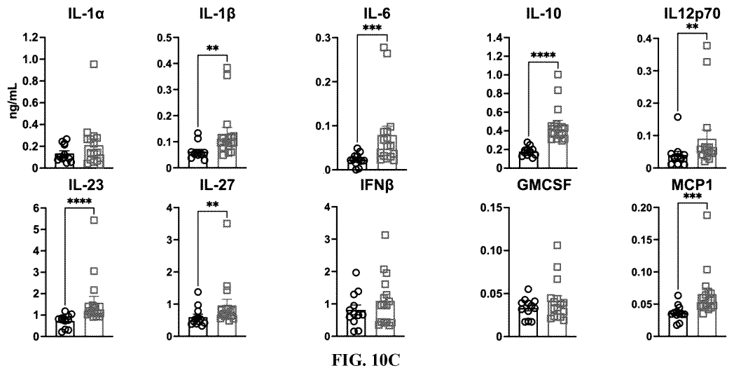

AIHA is a known side effect of ICPi immunotherapy(6, 7, 26). Considering this observation, together with genes identified by RNAseq, we tested whether tolerance could be reversed in young HOD⁺OTII⁺ mice by treating with blocking antibodies against checkpoint molecules PD1, Lag-3, and CTLA-4 as well as the immunosuppressive cytokine IL-10 receptor (IL-10R), denoted as “ICPi”. Within two weeks, ICPi-treated HOD⁺OTII⁺ mice had high levels of RBC autoantibodies, which continued to increase after treatment cessation (FIG. 2A). As in humans(27-29), female mice had higher autoantibody levels, compared to males (FIG. 2B). Autoantibodies were also detectable on RBCs from ICPi-treated mice, but not controls (FIG. 10A). ICPi treatment induced splenomegaly (not shown) and anemia, evidenced by significantly reduced hematocrit and hemoglobin levels, elevated serum bilirubin levels, and reticulocytosis (FIG. 2C and FIG. 10B); there was a significant correlation between disease severity and autoantibody levels (FIG. 2D). ICPi treatment stimulated production of multiple proinflammatory cytokines, including IFNγ, IL17, and TNF α (FIG. 2E and FIG. 10C)(30). Moreover, RBCs from autoantibody-producing mice exhibited elevated levels of detectable ROS, likely influencing AHIA pathogenesis (FIG. 2F)(31). Together, these data demonstrate that ICPi induces AIHA in our mouse model.

### Example 4—RBC Tolerance Reversal with ICPi is Generalizable

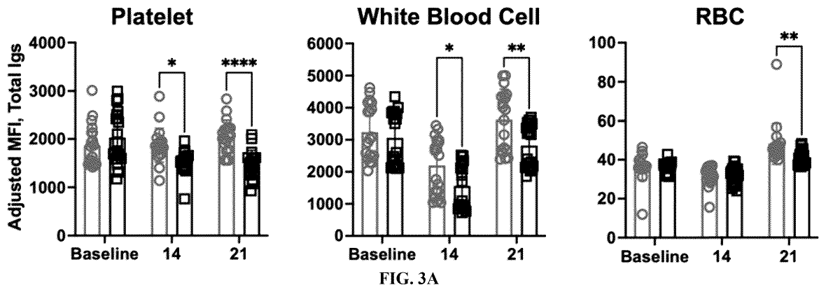

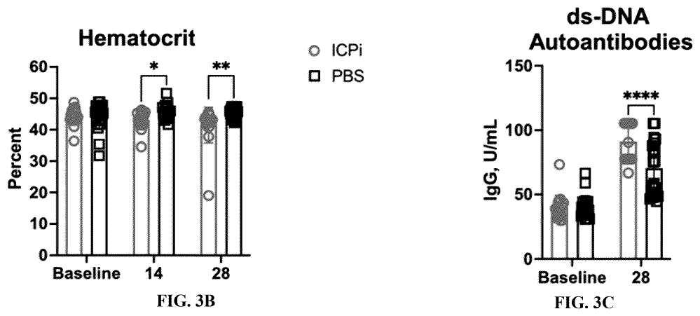

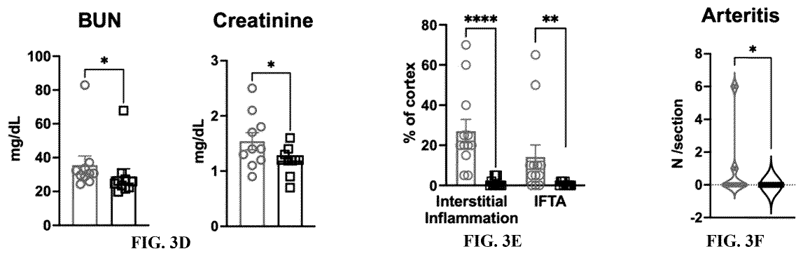

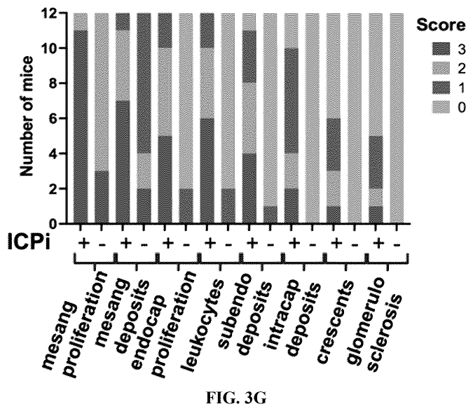

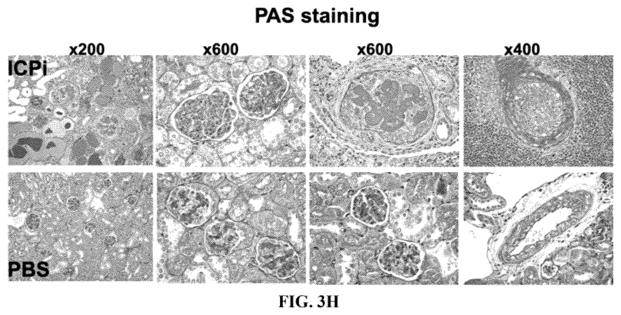

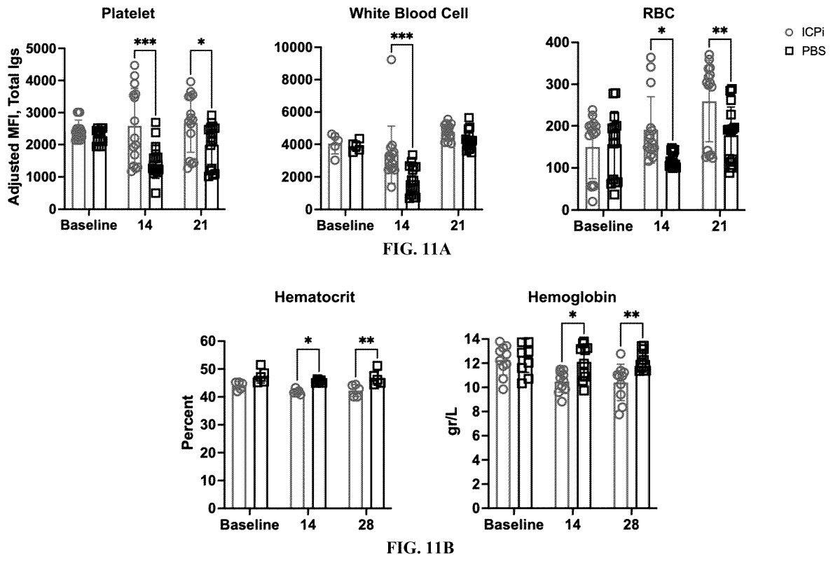

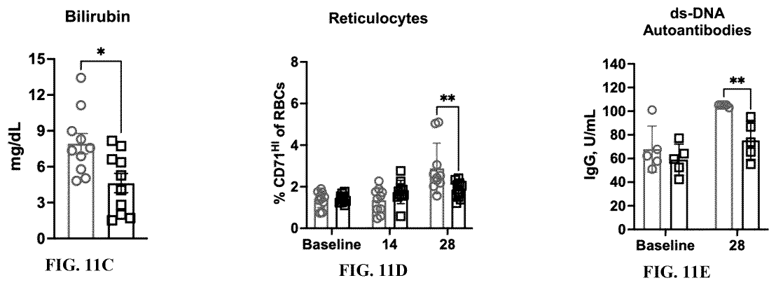

To test whether tolerance failure mechanisms for RBC autoantigens is shared by other widely-expressed autoantigens, NZB mice received ICPi; NZB mice are a well-characterized model of spontaneous development of human systemic lupus erythematosus (SLE) that produces age-onset autoantibodies reactive to anti-nuclear, platelet, and RBC autoantigens, as well as severe kidney disease at 6 months of age(32). Within two weeks, ICPi treatment promoted significant increases in detectable autoantibodies bound to peripheral platelets and white blood cells and, by three weeks, to RBCs (FIG. 3A). ICPi-treated NZB mice had significantly decreased hematocrit (FIG. 3B), and increased anti-dsDNA autoantibody levels, a hallmark of lupus progression (FIG. 3C). To assess whether ICPi treatment hastened other lupus features, serum and tissue were analyzed 3 months after treatment cessation. BUN and creatinine were significantly increased in ICPi-treated NZB mice (FIG. 3D), and PAS-stained kidney sections showed findings characteristic of severe active and progressive lupus nephritis, while control mice showed a mild phenotype (FIGS. 3E-3H). To test whether ICPi could facilitate tolerance failure in otherwise healthy animals, without a genetic predisposition to autoimmunity, B6 mice were treated with ICPi; similar kinetics of autoantibody production and reactivity were observed (FIG. 11). These data show that ICPi treatment accelerates autoimmunity in NZB mice and induces autoantibodies in healthy B6 animals, demonstrating a key role for these pathways in tolerance to multiple autoantigens expressed on various cell subsets and tissues.

### Example 5—ICPi Treatment Promotes Proinflammatory CD4⁺ T Cells

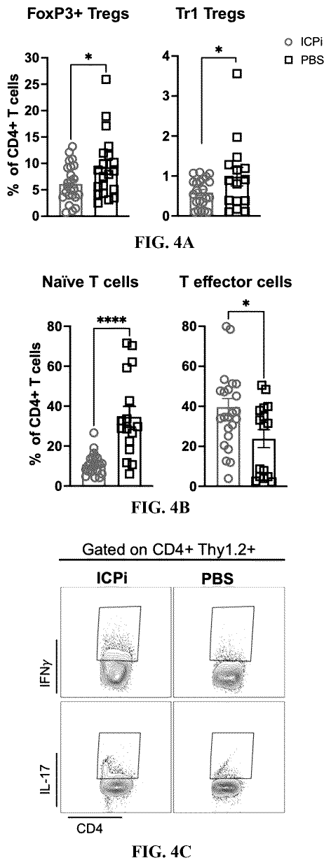

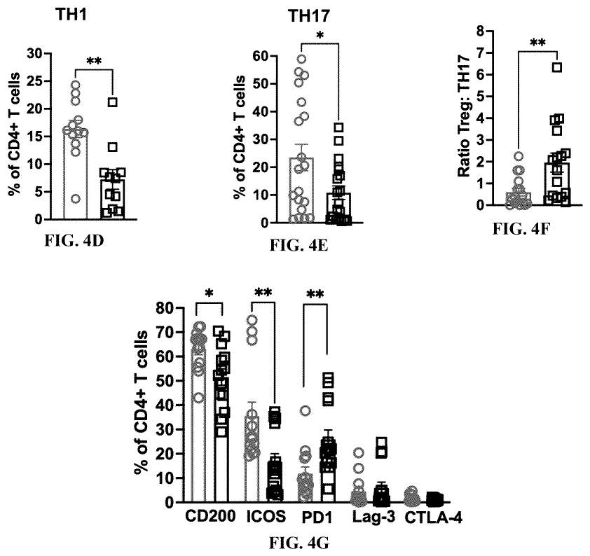

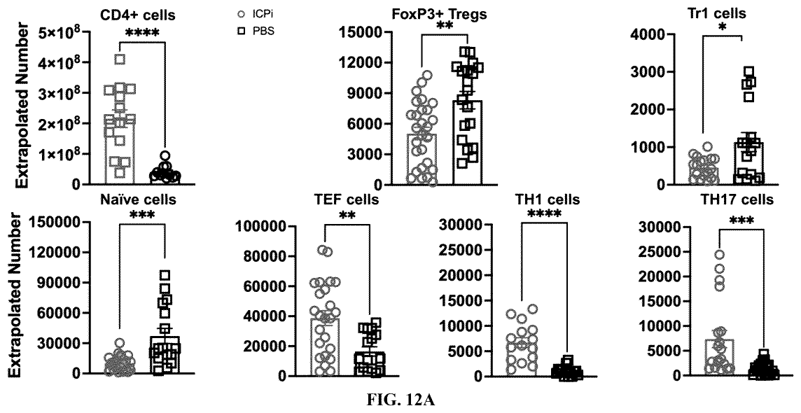

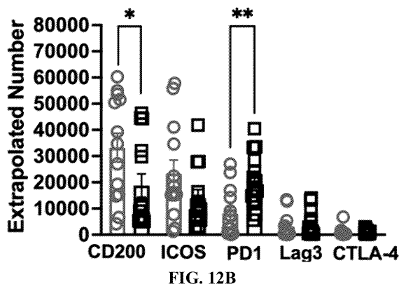

An imbalance between regulatory and proinflammatory T cells may initiate or perpetuate autoimmune diseases(30, 33-35). Consistent with prior findings, ~10% of CD4⁺ T cells in tolerized HOD⁺OTII⁺ mice were FoxP3⁺ Tregs(9), which were significantly reduced, alongside Tr Tregs, in ICPi-treated animals (FIG. 4A). Compared to controls, ICPi-treated mice had significantly decreased frequencies of naïve T cells and an expansion of effector T cells (FIG. 4B). To assess T helper cell subsets after ICPi treatment, enriched splenic CD4⁺ T cells were restimulated overnight in vitro. Increased frequencies of proinflammatory IFNγ+Th1 and IL17+Th17 T cells were observed in ICPi-treated animals, as compared to controls (FIGS. 4C-4E). Of note, the Treg:Th17 ratio was significantly decreased in ICPi-treated mice, demonstrating an imbalance between regulatory and proinflammatory T cells (FIG. 4F). Because ICPi-treated animals had splenomegaly, absolute CD4⁺ T cell numbers for each subset were extrapolated, which were significantly higher than controls (FIG. 12A).

In tumor immunotherapy, checkpoint molecule inhibition can induce compensatory upregulation of other immune molecules(36, 37). Therefore, enriched splenic CD4⁺ T cells were stained with antibodies against CD200, ICOS, and with non-competing clones for PD1, Lag-3, and CTLA-4 used in ICPi. In ICPi-treated mice, CD200 and ICOS expression were significantly increased, while PD1 was reduced (FIG. 4G and FIG. 12B). Lag-3 and CTLA-4 expression remained unchanged. Together, these data demonstrate dynamic modulation of checkpoint molecule expression in response to the ICPi.

### Example 6—CD39SP Predict RBC Tolerance Failure

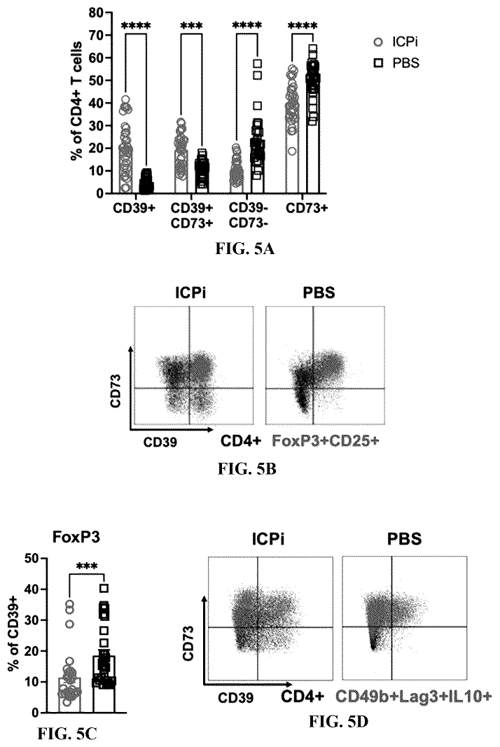

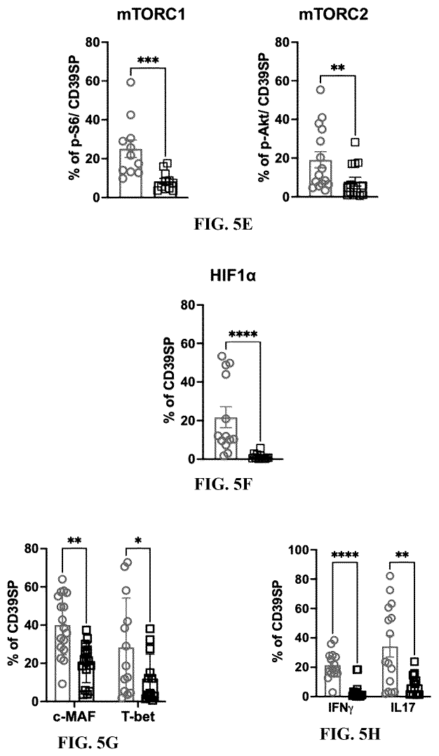

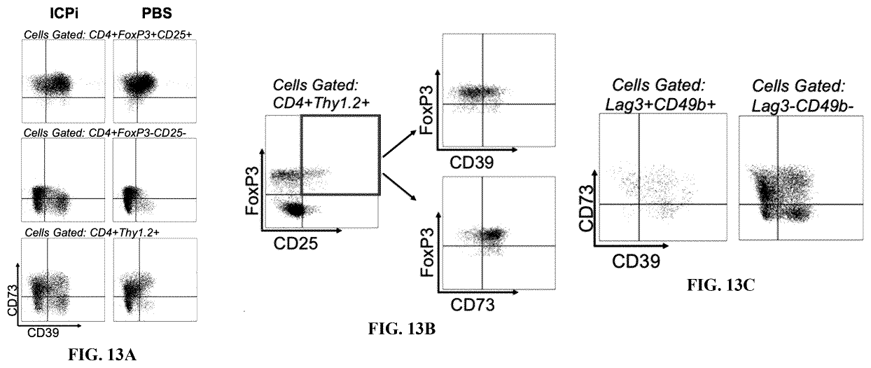

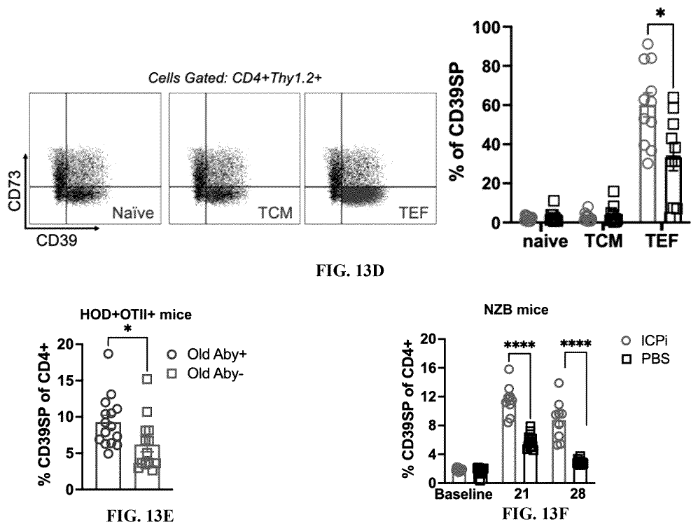

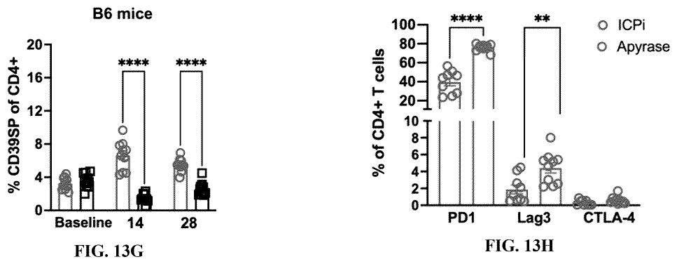

Based on the RNAseq data and the observed upregulation of surface CD73 expression in tolerized, autoreactive CD4⁺ RTEs, we hypothesized that purinergic signaling molecules are instrumental for RBC autoantigen tolerance. ICPi-treated mice had significant increases in frequencies of CD4⁺ T cells co-expressing CD39 and CD73, along with reductions in a subset expressing CD73 alone (FIG. 5A). However, a population of CD4⁺ T cells expressing only CD39 was significantly increased in ICPi-treated mice. As most Tregs co-express CD73 and CD39(20), we tested whether CD39⁺ were FoxP3⁺or Tr Tregs. While most FoxP3⁺ Tregs from ICPi-treated mice co-expressed CD39 and CD73, most CD39⁺ did not express FoxP3 (FIGS. 5B, 5C, 13A and 13B). Additionally, CD39⁺ lacked expression of Tr Treg markers Lag-3 and CD49b (FIGS. 5D and 13C). These data show that CD39⁺ do not express Treg markers and will be defined phenotypically as CD4⁺CD39⁺CD73⁻FoxP3⁻CD25⁻ and referred to as CD39SP.

To test whether CD39SP were metabolically active and determine their effector T cell subset, mTOR signaling molecules and cytokine production were evaluated. Compared to controls, CD39SP from ICPi-treated mice had significantly higher expression of p-S6 and p-Akt, markers of mTOR complex 1 and 2, respectively (FIG. 5E), and of HIF1α, indicating that CD39SP are metabolically active and may differentiate into Th17 T cells (FIG. 5F)(38). Moreover, CD39SP from ICPi-treated mice were more likely to express c-MAF and T-bet transcription factors and produce proinflammatory IFNγ and IL-17 upon re-stimulation (FIGS. 5G and 5H); no IL4 was detectable (not shown). CD39SP expressed CD62L^{high}CD44^{low}, a phenotype consistent with T effector cells (FIG. 13D). Together, these data suggest that CD39SP are pathogenic and may initiate and/or perpetuate AIHA.

Up to this point, the data support the concept that CD39SP from ICPi-treated mice exhibit effector/helper, rather than regulatory, T cell characteristics. To explore whether CD39SP were functional, their ability to provide B cell help (i.e., stimulating proliferation) was evaluated. CD39SP, which are specific for OVA, were sorted from ICPi-treated and naive HOD⁺OTII⁺ mice and co-cultured with HEL-specific transgenic B cells in the presence of soluble HEL or HEL+OVA. CD39SP from ICPi-treated animals, but not naïve, stimulated B cell proliferation when co-cultured with HEL+OVA, as inferred by decreased CellTrace-FR signal (FIG. 5I). No significant B cell proliferation was observed in B cells alone or with CD39SP from naïve mice. These data demonstrate that ICPi-induced CD39SP activate B cells, suggesting they may perpetuate AIHA disease.

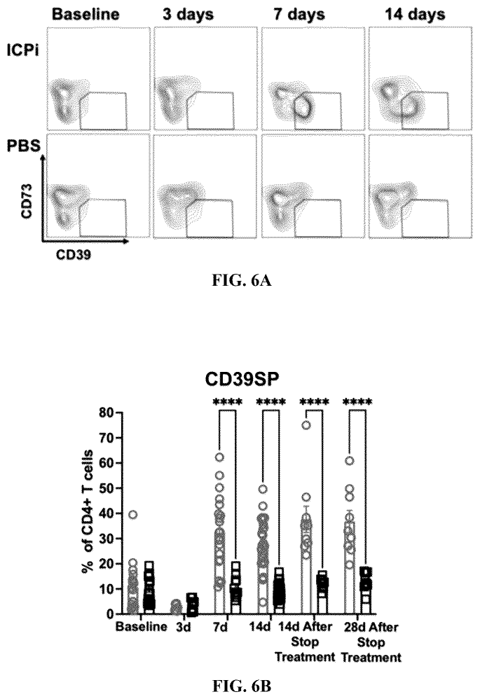

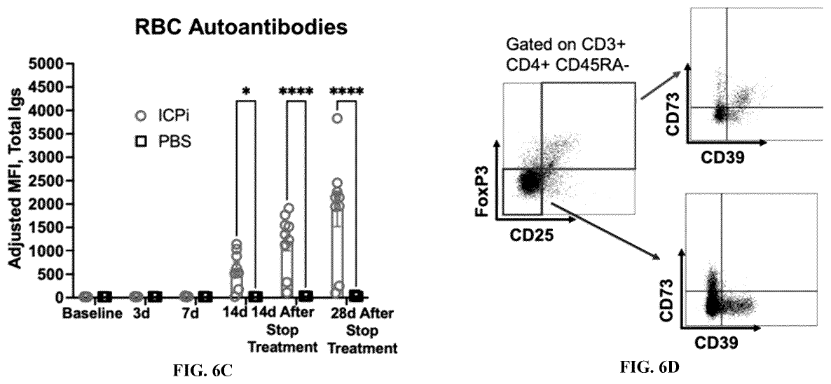

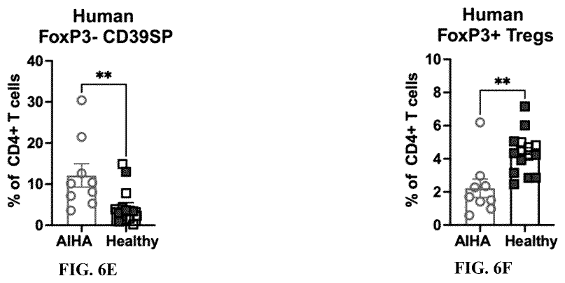

To gain insight into the role of CD39SP in AIHA pathogenesis, the kinetics of CD39SP appearance was monitored in peripheral blood during ICPi treatment. CD39SP appeared 7 days after ICPi treatment (FIG. 6A) and were detectable before RBC autoantibodies could be reliably identified (FIGS. 6B and 6C). Similar trends were noted in other models, including older HOD⁺OTII⁺ mice that had spontaneously produced autoantibodies with aging (FIG. 13E)(10), and in ICPi-treated NZB and B6 mice (FIGS. 13F and 13G), suggesting that CD39SP may be an early biomarker that predicts subsequent RBC autoantibody production.

As the role of purinergic signaling in AIHA has yet to be described in humans, we tested whether findings from our preclinical model could be extended to patients. Remarkably, CD39SP were detectable at higher frequencies in PBMCs from patients with AIHA compared to healthy controls (FIGS. 6D and 6E), while FoxP3⁺ Tregs were lower in patients (FIG. 6F), further supporting the importance of the purinergic signaling pathway in AIHA and overall translatability of our findings.

### Example 7—Apyrase Treatment Mitigates ICPi-Induced AIHA

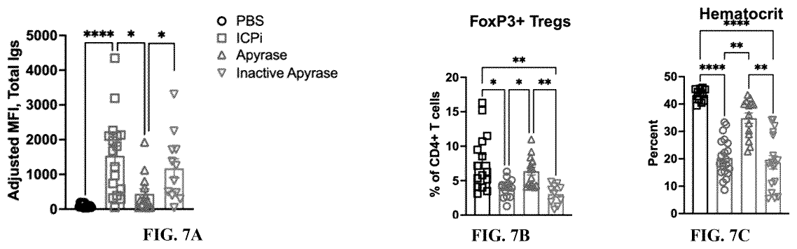

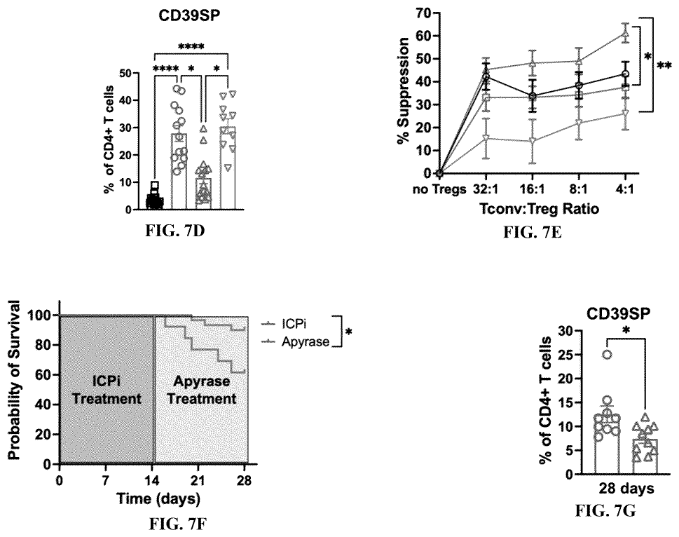

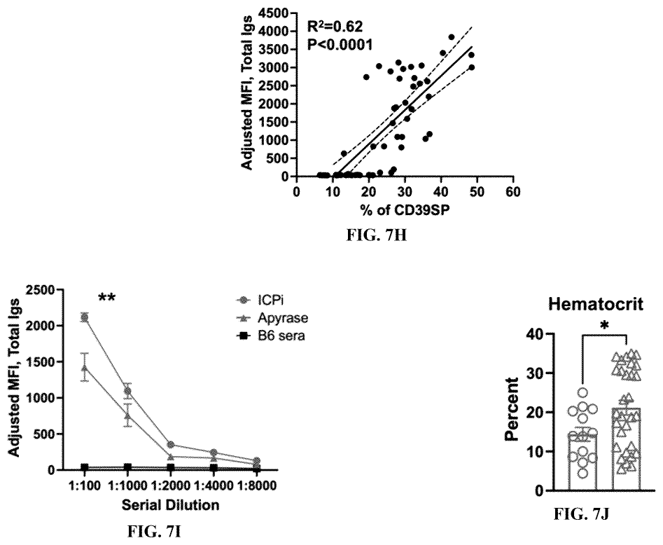

Extracellular ATP (eATP) is a proinflammatory signal and, together with decreased levels of immunosuppressive adenosine, can potentially induce autoimmunity(39). To test whether eATP plays a role in RBC autoantibody production, before exposure to ICPi, mice were prophylactically injected with apyrase, an enzyme that degrades ATP. Mice that received apyrase before every infusion of ICPi had significant reductions in RBC autoantibody levels and increased hematocrit, compared to controls (FIGS. 7A and 7B). This effect depended upon infusion of functional enzyme, as mice given heat-inactivated apyrase(14) produced RBC autoantibody levels comparable to mice treated with ICPi alone. Apyrase also prevented FoxP3⁺ Treg reductions (FIG. 7C) and blunted CD39SP expansion (FIG. 7D). Lastly, using an in vitro suppression assay, enriched CD4⁺CD25⁺ T cells from apyrase-treated mice exhibited a significantly increased capacity to suppress conventional T cells, as compared to T cells from animals treated with ICPi alone or with inactivated apyrase (FIG. 7E), which may be explained by upregulation of checkpoint molecule expression (FIG. 13H).

Apyrase was also evaluated therapeutically by treating mice with ongoing RBC autoantibody production (i.e., 14 days post-ICPi). While anemia and autoantibody levels were not ameliorated (not shown), apyrase treatment enhanced survival rates, compared to control mice given PBS (FIG. 7F). Like prophylactic administration, therapeutic apyrase led to significant reductions of CD39SP in ICPi-treated mice (FIG. 7G); CD39SP persisted at least 2 weeks ICPi and correlated with autoantibody levels (FIGS. 7G and 7H). Because increased CD39SP predicted tolerance failure and appeared before detectable RBC autoantibodies, we tested the effect of apyrase treatment at the time of CD39SP detection (but prior to autoantibody formation). Apyrase-treated mice had significant reductions in RBC autoantibody levels and increased hematocrit, compared to controls (FIGS. 71 and 7J). These findings demonstrate that reducing eATP levels with apyrase decreased AIHA development and augmented Treg function, suggesting a promising role for apyrase in attenuating ICPi-induced RBC autoantibodies and improving overall survival.

### Example 8—Discussion

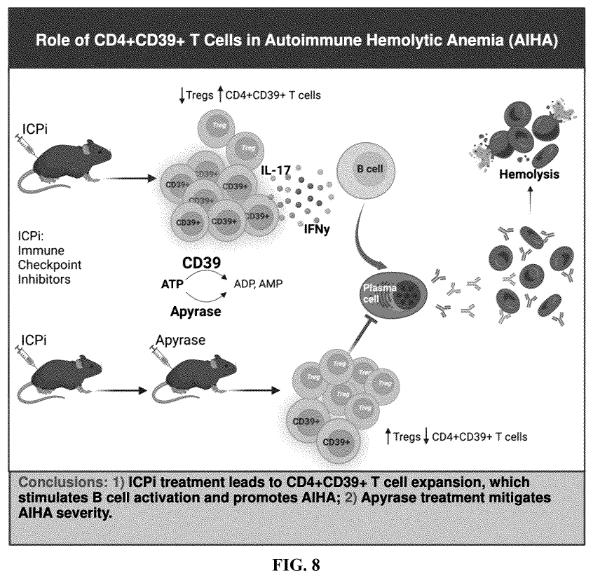

AIHA, both spontaneous and secondary to ICPi therapy, causes significant morbidity and mortality in humans. We recently reported that CD4⁺ T cell tolerance to RBC antigens occurs in the periphery, within 3 weeks after thymic egress(9), highlighting the crucial role of peripheral tolerance in preventing RBC autoimmunity. These findings were generated in a novel AIHA murine model that mirrors human idiopathic AIHA(10). Herein, we adapted this system to model ICPi-AIHA. This study advances understanding at three levels. First, we extended our previous correlative observations of specific immunoregulatory pathways to a direct causal role in preventing AIHA. Second, we established that ICPi-AIHA is preceded by, and associated with, increased CD39SP. Finally, we report that depriving CD39SP of its ATP substrate, with apyrase, mitigates ICPi-AIHA (FIG. 8). While these mechanistic advances were made in a mouse model, we confirmed an association of increased CD39SP and AIHA in humans. Increases in CD39SP may predict AIHA in humans and can be tested empirically in subsequent human studies.

Active suppression of autoimmunity has not been considered important in RBC tolerance, as CD25-mediated Treg depletion does not induce RBC autoantibodies in a mouse model(40). However, the current observation of two distinct Treg populations raises the possibility that FoxP3⁺ Tregs and Tr cells are redundant mechanisms of tolerance-neither being necessary to prevent AIHA, but either being sufficient. The importance of FoxP3⁺ Tregs in regulating human AIHA are unclear. Approximately 50% of humans with congenital FOXP3⁺ loss of function mutations (i.e., immunodysregulation, polyendocrinopathy, enteropathy, X linked [IPEX]) present with AIHA(41, 42). This suggests that FoxP3⁺ Tregs play an essential role in maintaining tolerance to RBC autoantigens in some humans, but not in all humans. Our data support a hypothesis that humans missing FoxP3⁺ Tregs without developing AIHA, have higher levels of compensatory Tr cells. Indeed, IPEX patients still generate Tr1(43, 44), and Tr have been proposed to have an important role in preventing AIHA. To our knowledge there are no available data to simultaneously assess FoxP3⁺ and Tr Treg levels in humans with AIHA, either in IPEX or due to other etiologies.

Proinflammatory Th1 and Th17 expansion was observed coincident with decreased FoxP3⁺ and Tr Tregs. Th17 play a crucial role in immune-mediated diseases and have links to AIHA severity in human patients and murine models(31, 35, 45, 46). In AHIA patients, FoxP3 downregulation correlates with decreased FoxP3⁺ Treg frequencies and expanded Th17 cells(30). Collectively, our data suggest that reducing two Treg subsets while increasing proinflammatory Th1 and Th17 cells, promotes AIHA. The origin of the Th1 and Th17 cells are unclear, but may include de novo generation and/or differentiation of Tregs into Th1 and Th17.

Tregs express high levels of CD39 and CD73. CD39 is an ectonuclease enzyme that, in cooperation with CD73, hydrolyzes eATP into adenosine(20). ATP can be pro-inflammatory, and chronically high eATP levels promote inflammation, inhibit FoxP3 expression, and can convert Tregs into Th17(46). In contrast, adenosine can be immunosuppressive, promote Treg generation, and prevent T cell activation and proliferation(25, 39). Thus, the co-expression of CD39 and CD73 facilitates conversion of proinflammatory eATP into immunosuppressive adenosine(20). Unexpectedly, a CD4⁺ T cell subset expressing CD39 without CD73 (i.e., CD39SP) emerged in ICPi-AIHA. Although we cannot rule out that CD39SP arose from unstable Tregs(47, 48), CD39SP lacked FoxP3 and Tr markers and expressed transcription factors and secreted cytokines most consistent with inflammatory Th1 and/or Th17 T cells.

CD39SP appeared before detectable RBC autoantibodies and stimulated B cell proliferation, suggesting they predict autoimmunity onset and may contribute to AIHA pathology. In addition to our ICPi-AIHA model, CD39SP were observed in our age-onset AIHA model, and in ICPi-treated NZB and healthy B6 mice, suggesting that CD39SP is a generalizable predictor of AIHA, regardless of etiology. Lastly, CD39SP were detectable in patients with secondary AIHA, without ICPi treatment, supporting the general association of CD39SP with RBC autoantibodies. Of note, patient samples were procured after AIHA diagnosis and medication administration, and additional studies are needed to evaluate the extent to which CD39SP predicts AIHA in humans.

The predictive power of CD39SP cells is made far more relevant considering the observation that apyrase was effective if administered at the time of CD39SP appearance, before autoantibody production. Importantly, while CD39SP frequencies persisted in untreated mice, they significantly decreased in apyrase-treated animals, demonstrating CD39SP are responsive to therapy and may provide a surrogate for therapeutic efficacy. We also detected a significant correlation between CD39SP and AIHA severity. Together, these data suggest that CD39SP analysis in PBMCs in patients undergoing ICPi therapy may enable early AIHA detection, facilitating timely intervention. Also, because current AIHA therapies have variable success(27-29), CD39SP monitoring may serve as an early predictor of potential treatment efficacy.

The current study has several limitations, particularly regarding the AIHA mouse model used, which has supraphysiological levels of RBC autoreactive CD4⁺ T cells, enabling study of antigen-specific responses. However, neither NZB nor B6 mice have supraphysiological levels of RBC autoreactive T cells, yet the same ICPi-AIHA was observed. Thus, it is unlikely that increased RBC autoreactive T cell frequency confounded the overall biology being studied. Second, our use of an ICPi cocktail targeting multiple checkpoints was designed to test causal roles of immunoregulatory molecules detected by unbiased screening. This differs from single-drug treatments, which have not been tested in this model; of note, tolerance fails within two weeks of treatment in these mice, compared to a 55-day average in patients receiving ICPi(49). Considering the complexity of multiple checkpoints and Treg populations, especially in the context of age- and sex-related factors influencing AIHA development, it is uncertain whether single-agent therapy would elicit as robust a response as the current combination, particularly in young animals. Finally, as with any animal model, there is the risk that the findings will not translate into human biology. However, CD39SP are detectable in PBMCs from patients with AIHA, suggesting at least an analogous cellular biology.

In summary, our studies introduce a novel mouse model of ICPi-AIHA, a well-documented irAE in cancer patients undergoing checkpoint inhibitor therapy. This model offers valuable mechanistic insights into tolerance breakdown, revealing a reduction in two distinct Treg subsets and an expansion of proinflammatory T cells. Our investigations unveil a previously unrecognized role for the purinergic signaling pathway in shaping RBC autoantigen tolerance, highlighting CD39SP as a predictive biomarker for tolerance failure and identifying apyrase as an effective therapeutic intervention. Furthermore, our findings extend beyond this model, as CD39SP are detectable in multiple mouse models developing AIHA and patients with AIHA, suggesting broad applicability to both idiopathic AIHA and AIHA secondary to other conditions.

### REFERENCES CITED AND INCORPORATED BY REFERENCE

1. Postow M A, Callahan M K, Wolchok J D. Immune Checkpoint Blockade in Cancer Therapy. J Clin Oncol. 2015; 33(17):1974-82.
2. Rowshanravan B, Halliday N, Sansom D M. CTLA-4: a moving target in immunotherapy. Blood. 2018; 131(1):58-67.
3. Thudium K, Selby M, Zorn J A, Rak G, Wang X T, Bunch R T, et al. Preclinical Characterization of Relatlimab, a Human LAG-3-Blocking Antibody, Alone or in Combination with Nivolumab. Cancer Immunol Res. 2022; 10(10):1175-89.
4. Martins F, Sofiya L, Sykiotis G P, Lamine F, Maillard M, Fraga M, et al. Adverse effects of immune-checkpoint inhibitors: epidemiology, management and surveillance. Nat Rev Clin Oncol. 2019; 16(9):563-80.
5. Khan U, Ali F, Khurram M S, Zaka A, Hadid T. Immunotherapy-associated autoimmune hemolytic anemia. J Immunother Cancer. 2017; 5:15.
6. Leaf R K, Ferreri C, Rangachari D, Mier J, Witteles W, Ansstas G, et al. Clinical and laboratory features of autoimmune hemolytic anemia associated with immune checkpoint inhibitors. Am J Hematol. 2019; 94(5):563-74.
7. Tanios G E, Doley P B, Munker R. Autoimmune hemolytic anemia associated with the use of immune checkpoint inhibitors for cancer: 68 cases from the Food and Drug Administration database and review. Eur J Haematol. 2019; 102(2):157-62.
8. Hudson K E, Hendrickson J E, Cadwell C M, Iwakoshi N N, Zimring J C. Partial tolerance of autoreactive B and T cells to erythrocyte-specific self-antigens in mice. Haematologica. 2012; 97(12):1836-44.
9. Wong ASL, Gruber D R, Richards A L, Sheldon K, Qiu A, Hay A, et al. Tolerization of recent thymic emigrants is required to prevent RBC-specific autoimmunity. J Autoimmun. 2020; 114:102489.
10. Dei Zotti F, Qiu A, La Carpia F, Moriconi C, Hudson K E. A New Murine Model of Primary Autoimmune Hemolytic Anemia (AIHA). Front Immunol. 2021; 12:752330.
11. Quintero O L, Amador-Patarroyo M J, Montoya-Ortiz G, Rojas-Villarraga A, Anaya J M. Autoimmune disease and gender: plausible mechanisms for the female predominance of autoimmunity. J Autoimmun. 2012; 38(2-3):J109-19.
12. Desmarets M, Cadwell C M, Peterson K R, Neades R, Zimring J C. Minor histocompatibility antigens on transfused leukoreduced units of red blood cells induce bone marrow transplant rejection in a mouse model. Blood. 2009; 114(11):2315-22.
13. Dei Zotti F, Moriconi C, Qiu A, Miller A, Hudson K E. Distinct CD4⁺ T cell signature in ANA-positive young adult patients. Front Immunol. 2022; 13:972127.
14. Coover R A, Healy T E, Guo L, Chaney K E, Hennigan R F, Thomson C S, et al. Tonic ATP-mediated growth suppression in peripheral nerve glia requires arrestin-PP2 and is evaded in NFl. Acta Neuropathol Commun. 2018; 6(1):127.
15. Thomas T A, Qiu A, Kim C Y, Gordy D E, Miller A, Tredicine M, et al. Reticulocytes in donor blood units enhance red blood cell alloimmunization. Haematologica. 2023; 108(10):2639-51.
16. Fink P J. The biology of recent thymic emigrants. Annu Rev Immunol. 2013; 31:31-50.
17. Collison L W, Vignali D A. In vitro Treg suppression assays. Methods Mol Biol. 2011; 707:21-37.
18. Crawford A, Angelosanto J M, Kao C, Doering T A, Odorizzi P M, Barnett B E, et al. Molecular and transcriptional basis of CD4(+) T cell dysfunction during chronic infection. Immunity. 2014; 40(2):289-302.
19. Gruarin P, Maglie S, De Simone M, Haringer B, Vasco C, Ranzani V, et al. Eomesodermin controls a unique differentiation program in human IL-10 and IFN-7 coproducing regulatory T cells. Eur J Immunol. 2019; 49(1):96-111.
20. Vignali D A, Collison L W, Workman C J. How regulatory T cells work. Nat Rev Immunol. 2008; 8(7):523-32.
21. Yuan X, Ferrari D, Mills T, Wang Y, Czopik A, Doursout M F, et al. Editorial: Purinergic Signaling and Inflammation. Front Immunol. 2021; 12:699069.
22. Freeborn R A, Strubbe S, Roncarolo M G. Type 1 regulatory T cell-mediated tolerance in health and disease. Front Immunol. 2022; 13:1032575.
23. Roncarolo M G, Gregori S, Bacchetta R, Battaglia M, Gagliani N. The Biology of T Regulatory Type 1 Cells and Their Therapeutic Application in Immune-Mediated Diseases. Immunity. 2018; 49(6):1004-19.
24. Zhang P, Lee J S, Gartlan K H, Schuster I S, Comerford I, Varelias A, et al. Eomesodermin promotes the development of type 1 regulatory T (T(R)1) cells. Sci Immunol. 2017; 2(10).
25. Bono M R, Fernandez D, Flores-Santibanez F, Rosemblatt M, Sauma D. CD73 and CD39 ectonucleotidases in T cell differentiation: Beyond immunosuppression. FEBS Lett. 2015; 589(22):3454-60.
26. Hwang S R, Saliba A N, Wolanskyj-Spinner A P. Immunotherapy-associated Autoimmune Hemolytic Anemia. Hematol Oncol Clin North Am. 2022; 36(2):365-80.
27. Barcellini W, Fattizzo B. Diagnosis and Management of Autoimmune Hemolytic Anemias. J Clin Med. 2022; 11(20).
28. Mulder F V M, Evers D, de Haas M, Cruijsen M J, Bernelot Moens S J, Barcellini W, et al. Severe autoimmune hemolytic anemia; epidemiology, clinical management, outcomes and knowledge gaps. Front Immunol. 2023; 14:1228142.
29. Tranekaer S, Hansen D L, Frederiksen H. Epidemiology of Secondary Warm Autoimmune Haemolytic Anaemia-A Systematic Review and Meta-Analysis. J Clin Med. 2021; 10(6).
30. Ciudad M, Ouandji S, Lamarthee B, Cladiere C, Ghesquiere T, Nivet M, et al. Regulatory T-cell dysfunctions are associated with increase in tumor necrosis factor alpha in autoimmune hemolytic anemia and participate in Th17 polarization. Haematologica. 2023.
31. Michalak S S, Olewicz-Gawlik A, Rupa-Matysek J, Wolny-Rokicka E, Nowakowska E, Gil L. Autoimmune hemolytic anemia: current knowledge and perspectives. Immun Ageing. 2020; 17(1):38.
32. Richard M L, Gilkeson G. Mouse models of lupus: what they tell us and what they don't. Lupus Sci Med. 2018; 5(1):e000199.
33. Grant C R, Liberal R, Mieli-Vergani G, Vergani D, Longhi M S. Regulatory T-cells in autoimmune diseases: challenges, controversies and—yet—unanswered questions. Autoimmun Rev. 2015; 14(2):105-16.
34. Noack M, Miossec P. Th17 and regulatory T cell balance in autoimmune and inflammatory diseases. Autoimmun Rev. 2014; 13(6):668-77.
35. Xu L, Zhang T, Liu Z, Li Q, Xu Z, Ren T. Critical role of Th17 cells in development of autoimmune hemolytic anemia. Exp Hematol. 2012; 40(12):994-1004 e4.
36. Fourcade J, Sun Z, Benallaoua M, Guillaume P, Luescher I F, Sander C, et al. Upregulation of Tim-3 and PD-1 expression is associated with tumor antigen-specific CD8⁺ T cell dysfunction in melanoma patients. J Exp Med. 2010; 207(10):2175-86.
37. Huang R Y, Francois A, McGray A R, Miliotto A, Odunsi K. Compensatory upregulation of PD-1, LAG-3, and CTLA-4 limits the efficacy of single-agent checkpoint blockade in metastatic ovarian cancer. Oncoimmunology. 2017; 6(1):e1249561.
38. Dang E V, Barbi J, Yang H Y, Jinasena D, Yu H, Zheng Y, et al. Control of T(H)17/T(reg) balance by hypoxia-inducible factor 1. Cell. 2011; 146(5):772-84.
39. Magni G, Ceruti S. Adenosine Signaling in Autoimmune Disorders. Pharmaceuticals (Basel). 2020; 13(9).
40. Richards A L, Kapp L M, Wang X, Howie H L, Hudson K E. Regulatory T Cells Are Dispensable for Tolerance to RBC Antigens. Front Immunol. 2016; 7:348.
41. Park J H, Lee K H, Jeon B, Ochs H D, Lee J S, Gee H Y, et al. Immune dysregulation, polyendocrinopathy, enteropathy, X-linked (IPEX) syndrome: A systematic review. Autoimmun Rev. 2020; 19(6):102526.
42. Wildin R S, Smyk-Pearson S, Filipovich A H. Clinical and molecular features of the immunodysregulation, polyendocrinopathy, enteropathy, X linked (IPEX) syndrome. J Med Genet. 2002; 39(8):537-45.
43. Hall A M, Vickers M A, Barker R N, Erwig L P. Helper T cells point the way to specific immunotherapy for autoimmune disease. Cardiovasc Hematol Disord Drug Targets. 2009; 9(3):159-66.
44. Passerini L, Di Nunzio S, Gregori S, Gambineri E, Cecconi M, Seidel M G, et al. Functional type 1 regulatory T cells develop regardless of FOXP3 mutations in patients with IPEX syndrome. Eur J Immunol. 2011; 41(4):1120-31.
45. Ahmad E, Elgohary T, Ibrahim H. Naturally occurring regulatory T cells and interleukins 10 and 12 in the pathogenesis of idiopathic warm autoimmune hemolytic anemia. J Investig Allergol Clin Immunol. 2011; 21(4):297-304.
46. Takenaka M C, Robson S, Quintana F J. Regulation of the T Cell Response by CD39. Trends Immunol. 2016; 37(7):427-39.
47. Wang J, Zhao X, Wan Y Y. Intricacies of TGF-beta signaling in Treg and Th17 cell biology. Cell Mol Immunol. 2023; 20(9):1002-22.
48. Zhou X, Bailey-Bucktrout S L, Jeker L T, Penaranda C, Martinez-Llordella M, Ashby M, et al. Instability of the transcription factor Foxp3 leads to the generation of pathogenic memory T cells in vivo. Nat Immunol. 2009; 10(9):1000-7.
49. Zhuang J, Du J, Guo X, Zhou J, Duan L, Qiu W, et al. Clinical diagnosis and treatment recommendations for immune checkpoint inhibitor-related hematological adverse events. Thorac Cancer. 2020; 11(3):799-804.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

I/We claim:

1. A method of determining the onset of an immune-related adverse event (irAE) in a subject, the method comprising:
providing a blood or serum sample from the subject;
determining the presence of cells in the blood or serum sample that express CD4 and CD39; and
determining the presence of blood cells that lack expression of CD73, CD25, and FoxP3 in the blood cells that express CD4 and CD39, wherein the presence of such blood cells indicates increased likelihood for the onset of an irAE.

2. The method of claim 1, further comprising determining the level of IL-17 and/or IFNγ secretion in the blood cells that express CD4 and CD39 and lack expression of CD73, CD25, and FoxP3 from the blood or serum sample, wherein secretion of IL-17 and/or IFNγ confirms increased likelihood for the onset of an irAE.

3. The method of claim 1, wherein the irAE is autoimmune hemolytic anemia.

4. A method of preventing the onset of or treating an immune-related adverse event (irAE) in a subject, the method comprising administering to the subject a therapeutic that degrades ATP.

5. The method of claim 4, wherein the therapeutic that degrades ATP is a purinergic signaling modulator.

6. The method of claim 5, wherein the purinergic signaling modulator is apyrase or a recombinant apyrase.

7. The method of claim 4, further comprising identifying the subject is at an increased likelihood of developing an irAE prior to administering apyrase.

8. The method of claim 7, wherein the step of identifying the subject is at an increased likelihood of developing an irAE comprises:
providing a blood or serum sample from the subject;
determining the presence of cells in the blood or serum sample that express CD4 and CD39; and
determining the presence of blood cells that lack expression of CD73, CD25, and FoxP3 in the blood cells that express CD4 and CD39, wherein the presence of such blood cells indicates increased likelihood for the onset of an irAE.

9. The method of claim 8, further comprising determining the level of IL-17 and/or IFNγ secretion in the blood cells that express CD4 and CD39 and lack expression of CD73, CD25, and FoxP3 from the blood or serum sample, wherein secretion of IL-17 and/or IFNγ confirms the subject is at an increased likelihood of developing an irAE.

10. The method of claim 4, wherein the irAE is autoimmune hemolytic anemia.

11. The method of claim 4, wherein the subject is administered an immune checkpoint inhibitor (ICPi) before, after, or concurrently with the therapeutic that degrades ATP.

12. The method of claim 11, wherein the ICPi is selected from the group consisting of anti-TIM3, anti-CTLA4, anti-PD-L1, anti-PD1, anti-TIGIT, and anti-LAG3.

13. A method of treating an immune-related adverse event (irAE) in a subject, the method comprising reducing the percentage of a subset of T cells positive for CD39 and negative for CD73, FoxP3, and CD25 in the subject.

14. The method of claim 13, wherein the subject is administered a therapeutic that degrades ATP thereby reducing the percentage of the subset of T cells positive for CD39 and negative for CD73, FoxP3, and CD25 in the subject.

15. The method of claim 14, wherein the therapeutic that degrades ATP is purinergic signaling modulator.

16. The method of claim 15, wherein the purinergic signaling modulatoris apyrase or a recombinant apyrase.

17. The method of claim 13, further comprising administering a steroid and/or rituximab to the subject.

18. The method of claim 13, wherein the irAE is autoimmune hemolytic anemia.

19. The method of claim 13, wherein the irAE is induced by inhibition of an immune checkpoint.

20. The method of claim 19, wherein the immune checkpoint is selected from the group consisting of: PD1 activation, Lag-3 activation, CTLA-4 activation, and IL-10 receptor activation.
